(19) United States
(12) Patent Application Publication
(10) Pub. No.: US 2018/0142290 A1
(43) Pub. Date: May 24, 2018

(54) BLOCKING OLIGONUCLEOTIDES

(71) Applicants: Kaarel Krjutskov, Huddinge (SE); Andres Salumets, Tartu (EE); Juha Kere, Stockholm (SE)

(72) Inventors: Kaarel Krjutskov, Huddinge (SE); Andres Salumets, Tartu (EE); Juha Kere, Stockholm (SE)

(21) Appl. No.: 15/577,261

(22) PCT Filed: Apr. 11, 2016
(86) PCT No.: PCT/SE2016/050303

§ 371 Date: Nov. 27, 2017

(30) Foreign Application Priority Data
May 28, 2015 (SE) 1550687-6

(51) Int. Cl. C12Q 1/6848; C12N 15/11; C12N 15/10

(52) U.S. Cl. CPC C12Q 1/6848; C12N 2310/34; C12N 15/1096; C12N 15/11

(57) ABSTRACT

A blocking oligonucleotide (10, 10A, 10A′, 10A″, 10B, 10C, 10D, 10E) comprises a 3′-end complementary sequence (13) complementary to a 3′-end sequence (23) of a globin mRNA molecule (20; 20A, 20B) and a poly-A complementary sequence (12) of at least one nucleotide complementary to at least a portion of a poly-A sequence (22) of the globin mRNA molecule (20, 20A, 20B). The blocking oligonucleotide (10, 10A, 10A′, 10A″, 10B, 10C, 10D, 10E) is capable of inhibiting binding of a reverse transcription anchored poly-T primer (30) to the globin mRNA molecule (20, 20A, 20B) and thereby significantly reducing synthesis of globin cDNA from globin mRNA molecules (20, 20A, 20B) present in a sample. This high reduction of globin cDNA by the blocking oligonucleotide (10, 10A, 10A′, 10A″, 10B, 10C, 10D, 10E) is achieved without any significant degradation of mRNA molecules present in the sample.

### TECHNICAL FIELD

The present embodiments generally relate to blocking oligonucleotides, and in particular such blocking oligonucleotides capable of targeted inhibition of reverse transcription of globin messenger ribonucleic acids, and uses thereof.

### BACKGROUND

In medical research, blood is a widely used biological sample source due to its high quality and quick dynamics reflecting an organism's response to a disease or specific treatment. For instance, blood, as a type of biopsy, is an important sample for research into 6,000 rare diseases and 12,000 disease groups. Although intravenous blood sampling is a somewhat invasive procedure, it is much more acceptable for donors in comparison to taking a solid tissue biopsy.

Whole blood ribonucleic acid (wbRNA) is widely used to carry out gene expression analysis by exploiting microarrays or massively parallel sequencing (RNA-seq) methods. Even though both approaches have high or even unlimited sensitivity, the enormous amount of globin messenger RNA (gmRNA) present in wbRNA samples have a strong inhibitory effect. This gmRNA leads to RNA-seq bias due to unbalanced, globin-dominated library. It has been shown that wbRNA consists of up to 50-80% globin alpha 1 and 2 (globin α1/2, HBA1/2) and globin beta (globin β, HBB) RNA molecules. Therefore, a conversion of gmRNA molecules into complementary deoxyribonucleic acid (cDNA) hampers a majority portion of cDNA synthesis power, simultaneously leaving biologically relevant mRNA molecules undetectable.

Thus, presence of gmRNA in a blood sample diminishes the scope of wbRNA usage, reduces sensitivity of RNA-seq methods and causes fold-change increase of sequencing costs in order to reach a desired coverage. Thus, presence of gmRNA is a severely limiting factor in wbRNA usage.

The GLOBINclear™-Human Kit (ThermoFisher) uses a non-enzymatic globin mRNA reduction technology that depletes >95% of the α and β gmRNA from total RNA preparations derived from whole blood. The kit uses long biotinylated oligonucleotides which hybridize specifically to gmRNA molecules. The resulting hybrids are then captured by streptavidin-coupled magnetic beads. The supernatant, i.e. the bulk-RNA with reduced gmRNA content, is further purified and enriched by clean-up procedures. The entire process takes approximately 90 min and needs labor work because it is designed for single-probe in single-tube format. A scientific report [1] demonstrates that when analyzing six RNAs, RNA integrity (RIN) value was decreased.

The GLOBINclear™ procedure is time-consuming and reduces RIN values (in 10 point scale) certainly 1-3 units, providing partly degraded RNA for cDNA synthesis. As shown herein, a RIN reduction of 11.8% (median) was detected in a 84-samples test and in 16 samples RIN values had reduced by more than 20%. As a result, even slightly fragmented mRNA samples cause a drop in gene 5′ detection rate to 54%, which in turn requires deeper sequencing depth and affect data quality in a way where shorter mRNA molecules are overrepresented and longer mRNA molecules are underrepresented due to mRNA degradation. GLOBINclear™ procedure further requires high amount of input RNA, typically 1-10 μg of human wbRNA, which is a limiting factor in case of rare and valuable samples.

Globin-Zero™ Gold kit from Epicentre (Illumina Inc.) seems to use ribonuclease H (RNase H) activity because the input RNA (1-5 μg) should be absolutely DNA and enzymatic inhibitors free. The outcome depends greatly on the RNA purity, making it far from a robust method.

ScriptSeg™ Complete Gold Kit (Epicentre, Illumina Inc.) provides a protocol similar to Globin-Zero™ but enables lower input RNA amounts (from 100 ng input wbRNA). ScriptSeg™ is based on using random hexamer reverse transcription priming during the cDNA synthesis.

A possible alternative to gmRNA reduction is to use peptide nucleic acid (PNA) oligonucleotides. PNA-based globin reduction is a non-enzymatic technology that silences the majority of α and β gmRNA molecules from total RNA preparations derived from whole blood. PNA oligomers can be effectively used as a clamp by specifically blocking gmRNA during the process of reverse transcription. PNAs can be also used as sequence specific PCR blockers because PNA probes have strong binding affinity and specificity to their target DNA and are not recognized by DNA polymerase as primer. Thus, PNAs have potential to reduce reverse transcription from gmRNAs and/or inhibit cDNA amplification from gmRNAs. GR PNA-L by Panagene are PNA oligonucleotides that specifically block gmRNA during the process of reverse transcription. However, with these PNA oligonucleotides reverse transcription of gmRNAs can be started but is stopped half-way due to the PNA-gmRNA double stand complex.

It is important to note that only PNA has silencing effect through specific hybridization with the target molecule. The other types of oligonucleotides, such as DNA, RNA, LNA, Zip nucleic acid (ZNA), etc., are removed by the strand replacement activity of reverse transcriptase and no significant reduction effect can thereby be achieved.

US 2006/0281092 relates to a process for the reverse transcription and/or amplification of a product from a reverse transcription of a pool of nucleic acids of a specific type. This pool of nucleic acids originates from a complex biological sample or an enzymatic reaction.

Thus, there is still a need for a technology that enables reduction of globin mRNA contamination in RNA samples, such as blood-extracted RNA samples.

### SUMMARY

It is a general objective to provide blocking oligonucleotides capable of inhibiting binding of reverse transcription anchored poly-T primers to globin mRNA molecules to thereby prevent or inhibit reverse transcription of globin mRNA molecules.

This and other objectives are met by embodiments as defined herein.

An aspect of the embodiments relates to a blocking oligonucleotide comprising, from a 3′-end towards a 5′-end of the blocking oligonucleotide, a 3′-end complementary sequence complementary to a 3′-end sequence of a globin mRNA molecule and a poly-A complementary sequence of at least one nucleotide complementary to at least a portion of a poly-A sequence of the globin mRNA molecule. The blocking oligonucleotide is capable of inhibiting binding of a reverse transcription anchored poly-T primer to the globin mRNA molecule.

Another aspect of the embodiments relates to a method of producing a complementary cDNA molecule. The method comprises contacting a sample comprising at least one mRNA molecule and at least one globin mRNA molecule with at least one blocking oligonucleotide as defined above under conditions enabling hybridization of a blocking oligonucleotide of the at least one blocking oligonucleotide to a globin mRNA molecule of the at least one globin mRNA molecule. The method also comprises adding a reverse transcription anchored poly-T primer and a reverse transcription enzyme to the sample to produce the cDNA molecule form the at least one mRNA molecule.

The blocking oligonucleotides of the embodiments are capable of reducing the prevalence of globin cDNA in a blood sample following reverse transcription from about 63% down to about 5% for human globins. This high reduction of globin cDNA by the blocking oligonucleotides is further achieved without any significant degradation of mRNA molecules, which is otherwise a common problem in the art.

### BRIEF DESCRIPTION OF THE DRAWINGS

The embodiments, together with further objects and advantages thereof, may best be understood by making reference to the following description taken together with the accompanying drawings, in which:

FIG. 1 schematically illustrates a globin mRNA molecule, possible binding site of anchored oligo-T primer, and blocking oligonucleotides according to various embodiments;

FIGS. 2A-2C illustrate secondary structures of globin mRNA HBA1 (A), HBA2 (B) and HBB (C) predicted by mFold software [7];

FIGS. 3A-3C illustrate secondary structures of globin mRNA HBA1 (A), HBA2 (B) and HBB (C) predicted by Vienna RNA software [8];

FIG. 4 illustrates the secondary structure of globin α1/2 (HBA1/2) and β (HBB) mRNA molecules and binding of blocking oligonucleotides according to embodiments to such globin α1/2 and β mRNA molecules;

FIG. 5 is a flow chart illustrating a method of producing a cDNA molecule;

FIG. 6 is a flow chart illustrating additional, optional steps of the method shown in FIG. 5;

FIG. 7A illustrates globin α1/2 mRNA reduction determined using quantitative polymerase chain reaction (qPCR);

FIG. 7B illustrates globin β mRNA reduction determined using qPCR;

FIGS. 7C and 7D illustrate cDNA yield change in the presence or absence of blocking oligonucleotide according to the embodiments determined by qPCR with two different artificial mRNA molecules (Spike 1 and Spike 2);

FIG. 7E illustrates absolute globin reduction fold change of globin α1/2 and β quantified by qPCR after treatment with blocking oligonucleotide according to the embodiments;

FIGS. 7F and 7G illustrate specificity of blocking oligonucleotide according to the embodiments measuring using two different artificial mRNA molecules (Spike 1 and Spike 2);

FIG. 8A illustrates globin α1/2 reduction analyzed using different concentrations of blocking oligonucleotide according to the embodiments;

FIG. 8B illustrates globin β reduction analyzed using different concentrations of blocking oligonucleotide according to the embodiments;

FIG. 9 illustrates globin α1/2 mRNA locking effect of five different blocking oligonucleotides measured by qPCR;

FIG. 10 illustrates the importance of the length of the poly-A complementary sequence of blocking oligonucleotides;

FIG. 11 graphically illustrates the specificity of different blocking oligonucleotides;

FIG. 12 illustrates the globin α1/2 mRNA locking effect at different target concentrations;

FIGS. 13A and 13B illustrate the globin α1/2 locking effect at different concentration of blocking oligonucleotide for HBA (A) and HBB (B);

FIG. 14 illustrates the globin reduction effect by different blocking oligonucleotides based on RNA sequencing experiment; and

FIG. 15 illustrates the fold reduction of the prevalence of globin mRNAs from different species by specific blocking oligonucleotides.

### DETAILED DESCRIPTION

Throughout the drawings, the same reference numbers are used for similar or corresponding elements. The present embodiments generally relate to blocking oligonucleotides, and in particular such oligonucleotides capable of inhibiting or reducing reverse transcription of globin messenger ribonucleic acids and uses thereof.

The blocking oligonucleotides of the embodiments can thereby be used in gene expression analysis, in which gene expression profiles are to be determined. The blocking oligonucleotides are in particular suitable in connection with gene expression analysis of whole blood RNA (wbRNA) and other biological samples containing large amounts of globin mRNA. Such high amounts of globin mRNA, such as up to about 50 to 80% of all RNA molecules in a typical wbRNA sample, will have a strong inhibitory effect in the gene expression analysis. Accordingly, there is a general need to prevent or at least reduce or inhibit reverse transcription of such globin mRNA molecules into cDNA during gene expression analysis. The blocking oligonucleotides of the embodiments are excellent tools that are capable of inhibiting or reducing the amount of globin mRNA that is reverse transcribed into cDNA by blocking or inhibiting binding of reverse transcription anchored poly-T primers to the globin mRNA molecule. As a consequence, the reverse transcription enzyme will have no start point to initiate reverse transcription of the globin mRNA since the reverse transcription anchored poly-T primer is prevented or at least inhibited from binding to the globin mRNA molecules.

The blocking oligonucleotides of the embodiments thereby utilizes a different mechanism to prevent or at least reduce reverse transcription of globin mRNA molecules as compared to antisense oligonucleotides known in the art, for instance in US 2006/0281092. Such prior art antisense oligonucleotides interrupt cDNA synthesis by stopping reverse transcription during synthesis. Thus, the antisense oligonucleotides do not block or prevent hybridization of reverse transcription primers to the globin mRNA molecule and thereby do not prevent start of reverse transcription. In clear contrast, the antisense oligonucleotides perform selective suppression of further nucleic acid polymerization, i.e. reverse transcription. This means that the polymerization interruption as described in US 2006/0281092 creates truncated cDNA molecules, thereby wasting enzyme activity and primers.

The suppressive effect is, however, only achieved using PNA as nucleotide species. The reason being that reverse transcription enzymes have well-known strand displacement activity. This means that after successful primer binding and during cDNA synthesis, the reverse transcription enzymes will remove any hindrances like a double-stranded region between the globin mRNA molecule and the antisense oligonucleotide on its way. The reverse transcription enzymes can, however, not displace the antisense oligonucleotides if they are made of PNA as compared to other nucleotide species, such as DNA, RNA or LNA.

The blocking oligonucleotides of the embodiments have a different mechanism and action as compared to these antisense oligonucleotides. The blocking oligonucleotides of the embodiments achieve a targeted inhibition of reverse transcription anchored poly-T primers to thereby prevent or at least inhibit binding of these primers to the globin mRNA molecules. Accordingly, the revers transcription enzymes, such as reverse transcriptase, will not have no free poly-T primer 3′ OH start site to initiate cDNA synthesis.

The selective inhibition of reverse transcription of globin mRNA molecules by the blocking oligonucleotides is achievable regardless of the nucleotide species of the blocking oligonucleotides. This means that the blocking oligonucleotides can be made of DNA, RNA, PNA and/or LNA nucleotides as illustrative but non-limiting examples and still achieve the desired blocking effect.

Experimental data as presented herein shows that blocking oligonucleotides of the embodiments are capable of reducing the prevalence of globin cDNA in a wbRNA sample following reverse transcription from about 63% down to about 5%. This high reduction of globin cDNA by the blocking oligonucleotides was further achieved without any significant degradation of mRNA molecules, which is otherwise a common problem in the art. For instance, GLOBINclear™ resulted in a significant mRNA degradation and only about 53% of all mRNA molecules in the blood sample were intact after treatment with GLOBINclear™ biotinylated oligonucleotides.

An aspect of the embodiments relates to a blocking oligonucleotide comprising, from a 3′-end towards a 5′-end of the blocking oligonucleotide, a 3′-end complementary sequence complementary to a 3′-end sequence of a globin mRNA molecule and a poly-A complementary sequence of at least one nucleotide complementary to at least a portion of a poly-A sequence of the globin mRNA molecule. The blocking oligonucleotide is capable of inhibiting binding of a reverse transcription anchored poly-T primer to the globin mRNA molecule.

The 3′-end complementary sequence of the blocking oligonucleotide is complementary to the 3′-end sequence of the globin mRNA molecule. This means that the 3′-end complementary sequence is capable of hybridizing or binding to the 3′-end sequence of the globin mRNA. Thus, base pairs are formed between nucleotides of the 3′-end complementary sequence and the 3′-end sequence to thereby form a duplex structure of the 3′-end complementary sequence and the 3′-end sequence.

Correspondingly, the poly-A complementary sequence, also referred to as poly-T or poly-U sequence herein, of the blocking oligonucleotide is complementary to at least a portion of the poly-A sequence or poly-A tail of the globin mRNA molecule. This means that the poly-A complementary sequence is capable of hybridizing or binding to a portion of the poly-A sequence of the globin mRNA molecule. Thus, base pairs are formed between nucleotides, preferably T or U, of the poly-A complementary sequence and a portion of the poly-A sequence to thereby form a duplex structure of the poly-A complementary sequence and a portion of the poly-A sequence.

The portion of the poly-A sequence that the poly-A complementary sequence binds to is preferably the first N nucleotides (A) of the poly-A sequence for a poly-A complementary sequence with a length of N nucleotides (T or U).

The blocking oligonucleotide thereby comprises, from a 3′-end towards a 5′-end of the blocking oligonucleotide, the 3′-end complementary sequence and the poly-A complementary sequence. In a preferred embodiment, the 5′-end of the 3′-end complementary sequence is connected to the 3′-end of the poly-A complementary sequence. Thus, the 3′-end complementary sequence and the poly-A complementary sequence are preferably interconnected forming a continuous oligonucleotide sequence.

Complementary to as used herein does not necessary mean that the complementary sequence needs to be 100% complementary to the target sequence. Hence, it is not necessary that each nucleotide in the complementary sequence is complementary to and base pair with the corresponding nucleotide in the target sequence.

In clear contrast, the important feature with regard to complementary is that the complementary sequence is capable of hybridizing and binding to the target sequence under conditions used during gene expression analysis experiments.

In a particular embodiment, complementary to implies that the complementary sequence is capable of selectively hybridizing to the target sequence. The term “selectively hybridizes” includes reference to hybridization, under stringent hybridization conditions, of a complementary sequence to a specific nucleic acid target sequence to a detectably greater degree, i.e. at least 2-fold over background, than its hybridization to non-target nucleic acid sequences and to the substantial exclusion of non-target nucleic acids. Selectively hybridizing sequences typically have about at least 40% complementary sequence identity, preferably at least 50% complementary sequence identifier, at least 60% complementary sequence identity, at least 70% complementary sequence identity, at least 80% complementary sequence identify, or at least 90% complementary sequence identity and most preferably 100% complementary sequence identity with each other.

The term “stringent hybridization conditions” include reference to conditions under which a complementary sequence will hybridize to its target sequence, to a detectably greater degree than other sequences, i.e. at least 2-fold over background. Stringent hybridization conditions are sequence-dependent and will be different in different circumstances. By controlling the stringency of the hybridization and/or washing conditions, target sequences can be identified which can be up to 100% complementary to the complementary sequence. Alternatively, stringency conditions can be adjusted to allow some mismatching in sequences so that lower degrees of similarity are detected.

“Stringent hybridization conditions” are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization conditions are selected to be about 5° C. lower than the thermal melting point (Tₘ) for the specific sequence at a defined ionic strength and pH.

The reverse transcription anchored poly-T primer that the blocking oligonucleotides of embodiments prevent or at least inhibit from binding to the globin mRNA molecule preferably comprises an anchored poly-T sequence that is complementary to and capable of hybridizing or binding to a poly-A sequence or tail. The reverse transcription anchored poly-T primer in addition comprises at least one additional selective nucleotide to define the “real start” of the mRNA, i.e. the portion of the mRNA excluding the poly-A tail. As is well known in the art, a typical eukaryotic, including human, mRNA typically contains, from a 5′-end to a 3′-end, a cap, a 5′ untranslated region (UTR), the coding sequence (CDS), a 3′ UTR and the poly-A tail. This means that the reverse transcription anchored poly-T primer preferably comprises at least one nucleotide that is complementary to the last nucleotide(s) in the 3′ UTR or, in the case the mRNA molecule lacks a 3′ UTR, to the last nucleotide(s) in the CDR in addition to the poly-A tail.

Non-limiting examples of such reverse transcription anchored poly-T primers have the general nucleotide sequence of:
3′-NV(T)_{P}-5′;
3′-V(T)ₚ-5′;
3′-NNV(T)ₚ-5′; or
3′-(N)_{Q}V(T)ₚ-5′, wherein

V is cytosine (C), adenine (A) or guanine (G), N is any nucleotide (C, A, G, thymidine (T) or uracil (U)), P is from 15 to 35, preferably from 20 to 30 and Q is from 0 to 6, preferably 1.

A typical example of reverse transcription anchored poly-T primer is:

(SEQ ID NO: 1)
3′-NVTTTTTTTTTTTTTTTTTTTTTTTTTTTTT-5′.

The purpose of the NV, V, NNV or (N)_{Q}V 3′-end of the reverse transcription anchored poly-T primer is to avoid random and multiple poly-T primings on poly-A tails, which may be up to 500 nucleotides long. As a consequence the reverse transcription anchored poly-T primer will bind to the 5′-end portion of poly-A tails since it includes at least one nucleotide that is complementary to the 3′-end of the 3′ UTR or the 3′-end of the CDS of the mRNA molecule.

Hence, a reverse transcription anchored poly-T primer as used herein is a poly-T primer with at least one non-T nucleotide at its 3′-end, i.e. at the portion downstream (when going in the 5′ to 3′ direction) of the poly-T part.

The blocking oligonucleotides of the embodiments bind to at least the 3′-end sequence and a portion of the poly-A tail of globin mRNA molecules. Due to this binding the blocking oligonucleotides thereby prevent or at least significantly reduce the binding of reverse transcription anchored poly-T primers to the globin mRNA molecules. Accordingly, when a reverse transcription enzyme is added there will be no or at least significantly fewer reverse transcription anchored poly-T primers bound to globin mRNA molecules. The reverse transcription enzyme thereby has no primer free 3′ OH start site to initiate the reverse transcription from for the globin mRNA molecules. This implies that no or reduced amount of global cDNA molecules will be produced.

In an embodiment, a 3′-end of the blocking oligonucleotide is chemically modified to inhibit enzymatic extension of the blocking oligonucleotide. Thus, the 3′-end of the blocking oligonucleotide preferably has a chemical modification that prevents or inhibits extension of the blocking oligonucleotide during reverse transcription. This chemical modification of the 3′-end of the blocking oligonucleotide prevents the blocking oligonucleotide from being a reverse transcription primer for the reverse transcription enzyme.

Any chemical modification that prevents enzymatic extension of the blocking oligonucleotide in the presence of a reverse transcription enzyme but still allows the blocking oligonucleotide to bind to at least the 3′-end of the global mRNA molecule can be used according to the embodiments. A non-limiting but preferred example of such chemical modification is phosphorylation. Hence, the 3′-end of the blocking oligonucleotide is preferably phosphorylated. Another example of chemical modification is to have a Zip Nucleic Acid (ZNA) nucleotide at the 3′-end.

In an embodiment, the 3′-end complementary sequence consists of 10 to 100 nucleotides complementary to the 3′-end sequence of the global mRNA molecule. Preferably, the 3′-end complementary sequence consists of 15 to 60, more preferably 20 to 40, such as 25 to 35 or 27 to 32 nucleotides complementary to the 3′-end sequence of the global mRNA molecule.

In an embodiment, the poly-A complementary sequence comprises at least two nucleotides, preferably at least three nucleotides and more preferably at least four nucleotides complementary to at least a portion of the poly-A sequence of the globin mRNA molecule.

Experimental data as presented herein indicate that a poly-A complementary sequence preferably comprises at least four nucleotides if the blocking oligonucleotide is made of DNA. However, if at least some of the nucleotides of the blocking oligonucleotide are, for instance, LNA nucleotides the blocking oligonucleotide will be “sticky” and enabling formation of stable globin mRNA—blocking oligonucleotide complexes. In such cases, the number of nucleotides in the poly-A complementary sequence may be fewer than four and still achieve the desired targeted inhibition of reverse transcription of globin mRNA molecules.

In an embodiment, the poly-A complementary sequence consists of 1 to 50 nucleotides complementary to the at least a portion of the poly-A sequence of the globin mRNA molecule. Preferably, the poly-A complementary sequence consists of 1 to 30, such as 2 to 30, and more preferably 4 to 20 nucleotides complementary to the at least a portion of the poly-A sequence of the globin mRNA molecule. In a particular embodiment, the poly-A complementary sequence consists of 10 to 20, more preferably 13 to 16 nucleotides complementary to the at least a portion of the poly-A sequence of the globin mRNA molecule.

The nucleotide(s) of the poly-A complementary sequence is (are) preferably T.

In a particular embodiment, the 3′-end complementary sequence has the following nucleotide sequence 5′-GCYGCCCACTCAGACTTTATTCAAAGAC-3′ (SEQ ID NO: 2), wherein Y denotes T or C. Such a 3′-end complementary sequence is complementary to the 3′-end sequence of human globin α (HBA) mRNA molecules. In a particular embodiment, the globin α mRNA molecule is a human globin α1 (HBA1) mRNA molecule and Y is C. In another particular embodiment, the globin α mRNA molecule is a human globin α2 (HBA2) mRNA molecule and Y is T. The sequence shown above and in SEQ ID NO: 2 is universal for HBA1 and HBA2.

In another particular embodiment, the 3′-end complementary sequence has the following nucleotide sequence 5′-GCAATGAAAATAAATGTTTTTTATTAGGCAG-3′ (SEQ ID NO: 3). Such a 3′-end complementary sequence is complementary to the 3′-end sequence of human globin β (HBB) mRNA molecules.

In an embodiment, the blocking oligonucleotide comprises, or consists of, the following nucleotide sequence 5′-TTTTTTTGCYGCCCACTCAGACTTTA-3′ (SEQ ID NO: 82), wherein Y denotes T or C. Such a blocking oligonucleotide is complementary to HBA mRNA molecules. In a particular embodiment, the HBA mRNA molecule is a HBA1 mRNA molecule and Y is C. In another particular embodiment, the HBA mRNA molecule is a HBA2 mRNA molecule and Y is T.

In a related embodiment, the blocking oligonucleotide comprises, or consists of, the following nucleotide sequence 5′-TTTTTTTTTTTTGCAATGAAAATAAATGTTTTTTATTAGG-3′ (SEQ ID NO: 85). Such a blocking oligonucleotide is complementary to HBB mRNA molecules.

Such blocking oligonucleotides mentioned above and found in SEQ ID NO: 82, 85 are generally denoted 3′ end DNA short blocking oligonucleotides to denote that they have a comparatively short 3′-end complementary sequence.

In another embodiment, the blocking oligonucleotide comprises, or consists of, the following nucleotide sequence 5′-TTTTTTTTTTTTTTTGCYGCCCACTCAGACTTTATTCAAAGACCA-3′ (SEQ ID NO: 84), wherein Y denotes T or C. Such a blocking oligonucleotide is complementary to HBA mRNA molecules. In a particular embodiment, the HBA mRNA molecule is a HBA1 mRNA molecule and Y is C. In another particular embodiment, the HBA mRNA molecule is a HBA2 mRNA molecule and Y is T.

In another related embodiment, the blocking oligonucleotide comprises, or consists of, the following nucleotide sequence 5′-TTTTTTTTTTTTTTTGCAATGAAAATAAATGTTTTTTATTAGGCAGAATCCAGAT-3′ (SEQ ID NO: 87). Such a blocking oligonucleotide is complementary to HBB mRNA molecules.

Such blocking oligonucleotides mentioned above and found in SEQ ID NO: 84, 87 are generally denoted 3′ end DNA long blocking oligonucleotides to denote that they have a comparatively long 3′-end complementary sequence.

In a further embodiment, the globin mRNA molecule is a globin α mRNA molecule and the blocking oligonucleotide comprises, or consists of, the sequence of: 5′-TTTTTTTTTTTTTTTGCYGCCCACTCAGACTTTATTCAAAGAC-3′ (SEQ ID NO: 4),

wherein Y denotes T or C. In a particular embodiment, the globin α mRNA molecule is a globin α1 mRNA molecule and Y is C. In another particular embodiment, the globin α mRNA molecule is a globin α2 mRNA molecule and Y is T.

In a further related embodiment, the globin mRNA molecule is a globin β mRNA molecule and the blocking oligonucleotide comprises, or consists of, the sequence of:

(SEQ ID NO: 5)
5′-TTTTTTTTTTTTTTTGCAATGAAAATAAATGTTTTTTATTAGGC
AG-3′
or
(SEQ ID NO: 85)
5′-TTTTTTTTTTTTGCAATGAAAATAAATGTTTTTTATTAGG-3′.

Such blocking oligonucleotides mentioned above and found in SEQ ID NO: 4, 85 are generally denoted 3′ ZNA blocking oligonucleotides or 3′ ZNA-modified DNA blocking oligonucleotides if the 3′-end of the blocking oligonucleotides have a ZNA-modified oligonucleotide.

The nucleotides of the blocking oligonucleotides of the embodiments could be deoxy ribonucleotides (A, T, G, C), i.e. the blocking oligonucleotides are DNA molecules, or ribonucleotides (A, U, G, C), i.e. the blocking oligonucleotides are RNA molecules. It is also possible to use nucleic acid analogues in the blocking oligonucleotides including, for instance, peptide nucleic acid (PNA), locked nucleic acid (LNA), glycol nucleic acid (GNA) and threose nucleic acid (TNA).

Thus, the blocking oligonucleotides can comprise any nucleotides, natural and/or artificial, as long as the blocking oligonucleotides are capable of exerting their intended function as described herein, i.e. inhibit binding of reverse transcription anchored poly-T primers to globin mRNA molecules.

In an embodiment, the blocking oligonucleotide, preferably the 3′-end complementary sequence of the blocking oligonucleotide, comprises at least one LNA nucleotide. In a particular embodiment, the 3′-end complementary sequence comprises at least two, at least three, at least four or more LNA nucleotides.

In yet another embodiment, the globin mRNA molecule is a globin α mRNA molecule and the blocking oligonucleotide comprises, or consists of, the sequence of: 5′-TTTTTG+CYGCCC+ACTCAG+ACTTTA+TTC-3′ (SEQ ID NO: 83), wherein +C, +A and +T denote LNA nucleotides, wherein Y denotes T or C. In a particular embodiment, the globin α mRNA molecule is a globin α1 mRNA molecule and Y is C. In another particular embodiment, the globin α mRNA molecule is a globin α2 mRNA molecule and Y is T.

In yet another related embodiment, the globin mRNA molecule is a globin β mRNA molecule and the blocking oligonucleotide comprises, or consists of, the sequence of: 5′-TTTTTTTTTTG+CAATGA+AAATAA+ATGTTT+TTTAT TAGG-3′ (SEQ ID NO: 86), wherein +C, +A and +T denote LNA nucleotides.

Such blocking oligonucleotides mentioned above and found in SEQ ID NO: 83, 86 are generally denoted 3′ LNA blocking oligonucleotides to denote that the 3′-end complementary sequence comprises at least one LNA nucleotide.

The sequence examples of blocking oligonucleotides and sub-sequences thereof presented herein comprise the nucleotides A, T, G and C. The invention also encompasses corresponding blocking oligonucleotides comprising the nucleotides A, U, G and C. This corresponds to the replacing any thymidines (T) in the presented nucleotide sequences of the blocking oligonucleotides with uracils (U).

In an embodiment, the blocking oligonucleotide comprises a 5′-end complementary sequence complementary to a 5′-end sequence of the globin mRNA molecule and a linker sequence.

In such an embodiment, the blocking oligonucleotide is capable of binding to both the 5′-end sequence, a portion of the poly-A sequence and the 3′-end sequence of the globin mRNA molecule. The linker sequence preferably interconnects the 5′-end complementary sequence and the poly-A complementary sequence. As a consequence, the blocking oligonucleotide and the globin mRNA molecule form a circular complex when the 5′-end complementary sequence is hybridized to the 5′-end sequence of the globin mRNA molecule, the poly-A complementary sequence is hybridized to a portion of the poly-A sequence of the globin mRNA molecule and the 3′-end complementary sequence is hybridized to the 3′-end sequence of the globin mRNA molecule.

In an embodiment, the blocking oligonucleotide comprises, or consists of, from a 5′-end towards a 3′-end of the blocking oligonucleotide, the 5′-end complementary sequence, the linker sequence, the poly-A complementary sequence and the 3′-end complementary sequence.

In an embodiment, the 5′-end complementary sequence consists of 10 to 40 nucleotides complementary to the 5′-end sequence of the globin mRNA molecule. In a preferred embodiment, the 5′-end complementary sequence consists of 15 to 35, preferably 20 to 30 and more preferably 25 to 30 nucleotides complementary to the 5′-end sequence of the globin mRNA sequence.

In an embodiment, the 5′-end complementary sequence has the following nucleotide sequence 5′-CGCGAGCGCGCCAGGGTTTATG-3′ (SEQ ID NO: 6). This 5′-end complementary sequence is adapted for hybridization to the 5′-end sequence of a globin α1/2 mRNA molecule. In another embodiment, the 5′-end complementary sequence has the following nucleotide sequence 5′-AGTGAACACAGTTGTGTCAGAAGCAAATGT-3′ (SEQ ID NO: 7). This 5′-end complementary sequence is adapted for hybridization to the 5′-end sequence of a globin β mRNA molecule.

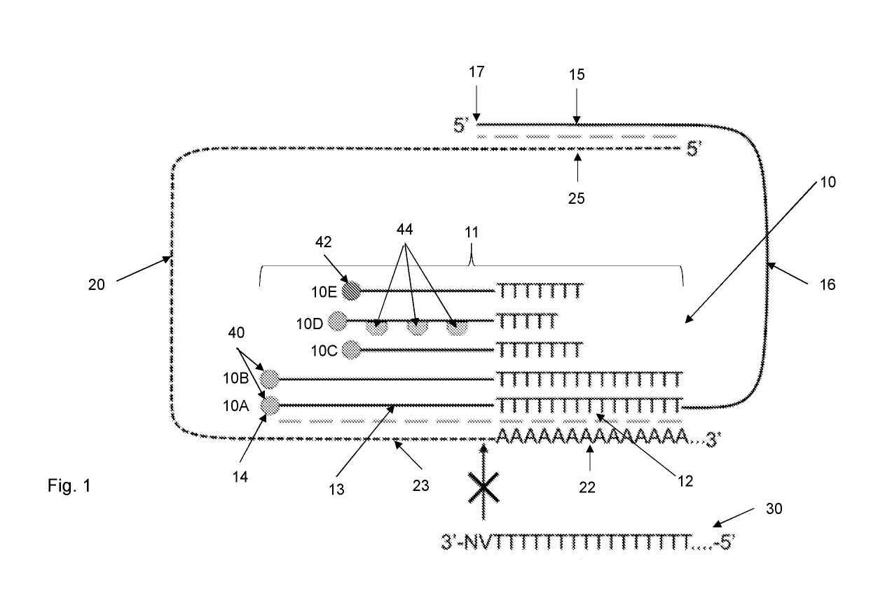

In an embodiment, the linker sequence consists of 30 to 80 nucleotides, preferably 40 to 60, such as 40 to 55 nucleotides. Such a length of the linker sequence enables the 5′-end and 3′-end complementary sequences of the blocking oligonucleotide to hybridize to the 5′-end and 3′-end sequences of the globin mRNA molecule, respectively, to form possible circular complex as shown in FIG. 1.

The linker sequence can generally have any nucleotide sequence since it should not hybridize or bind to any specific part of the globin mRNA molecule. Thus, in an embodiment, the linker sequence is preferably not complementary to any sequence of the globin mRNA molecule. This means that the linker sequence should preferably not bind to the globin mRNA molecule. In addition, the linker sequence should preferably prevent self-binding of the blocking oligonucleotide. Hence, the linker sequence is preferably not complementary to the 5′-end complementary sequence, the 3′-end complementary sequence or the poly-A complementary sequence of the blocking oligonucleotide. The linker sequence furthermore preferably has a nucleotide sequence selected to prevent self-binding of the linker sequence to itself.

Blocking oligonucleotides comprising a 3′-end complementary sequence, a poly-A complementary sequence, a linker sequence and a 4′-end complementary sequence are generally denoted 3′-5′ end DNA blocking oligonucleotides herein. Examples of such blocking oligonucleotides are found in SEQ ID NO: 8, 10.

FIG. 1 schematically illustrates a blocking oligonucleotide 10 according to various embodiments, generally denoted 10A to 10 E in the figure, hybridized to a globin mRNA molecule 20. The first blocking oligonucleotide 10A, denoted 3′-5′ ends DNA herein, has a 5′-end complementary sequence 15 hybridized to the 5′-end sequence 25 of the globin mRNA molecule 20. The 3′-end complementary sequence 13 is correspondingly hybridized to the 3′-end sequence 23 of the globin mRNA molecule 20. The poly-A complementary sequence 12 that constitutes together with the 3′-end complementary sequence 13 a 3′-portion 11 of the blocking oligonucleotide 10A is in turn hybridized to at least a portion of the poly-A sequence or tail 22 of the globin mRNA molecule 20. In this embodiment, the linker sequence 16 interconnects the 5′-end complementary sequence 15 and the poly-A complementary sequence 12.

Accordingly, in an embodiment the blocking oligonucleotide 10A comprises, from a 5′-end 17 towards a 3′-end 14 of the blocking oligonucleotide 10, the 5′-end complementary sequence 15, the linker sequence 16, the poly-A complementary sequence 12 and the 3′-end complementary sequence 13. The figure shows the circular complex formed by the first blocking oligonucleotide 10A and the globin mRNA molecule 20.

A second blocking oligonucleotide 10B, denoted 3′ end DNA long, lacks the linker sequence 16 and the 5′-end complementary sequence 15. In the figure, the second blocking oligonucleotide 10B has the same lengths of the 3′-end complementary sequence 13 and the poly-A complementary sequence 12 as the first blocking oligonucleotide 10A. However, the embodiments are not limited thereto. For instance, a third blocking oligonucleotide 10C, denoted 3′ end DNA short, has a comparatively shorter 3′-end complementary sequence 13 and poly-A complementary sequence 12 as compared to the first blocking oligonucleotide 10A and the second blocking oligonucleotide.

A fourth blocking oligonucleotide 10D, denoted 3′ LNA, comprises LNA nucleotides 44 in the 3′-end complementary sequence 13.

FIG. 1 schematically illustrates the chemical modification 40, 42 of the 3′-end 14 of the blocking oligonucleotides 10. In an embodiment, the chemical modification is a phosphorylation 40 of the 3′-end 14 of the first to fourth blocking oligonucleotides 10A-10D. The figure also illustrates a fifth blocking oligonucleotide 10E, denoted 3′ ZNA, that comprises a ZNA nucleotide 42 at its 3′-end 14.

When the blocking oligonucleotides 10, 10A-10E are hybridized to the globin mRNA molecule 20 as schematically shown in FIG. 1, the blocking oligonucleotides 10 inhibit binding of the reverse transcription anchored poly-T primer 30 to the globin mRNA molecule 20.

In an embodiment, the blocking oligonucleotide 10A comprises, preferably consists of, the sequence of 5′-CGCGAGCGCGCCAGGGTTTATG(Xₙ)_{N}TTTTTTTTTTTTTTTGCYGCCCACTCAGACTTTATTCAAA GAC-3′, wherein Xₙ is A, T, G or C, n=1-N and N is from 40 to 60. In a preferred embodiment, the blocking oligonucleotide 10A comprises, preferably consists of, the sequence of 5′-CGCGAGCGCGCCAGGGTTT ATGTAATTAGAATTAGAATGAATAGCTAACCTGATATGTTGAAGAACTATGACAGACATTTTTTTTTT TTTTTGCYGCCCACTCAGACTTTATTCAAAGAC-3 (SEQ ID NO: 8)′, and more preferably the sequence of 5′-CGCGAGCGCGCCAGGGTTTATGTAATTAGAATTAGAATGAATAGCTAACCTGATAT GTTGAAGAACTATGACAGACATTTTTTTTTTTTTTTGCYGCCCACTCAGACTTTATTCAAAGAC-Pho-3′ (SEQ ID NO: 9), wherein Pho denotes phosphorylation. Such blocking oligonucleotides are designed to bind to globin α mRNA molecules. As previously mentioned if the globin α mRNA molecule is a globin α1 mRNA molecule then Y is C and if the globin α mRNA molecule is a globin α2 mRNA molecule (20; 20A) and Y is T.

Further preferred embodiments of the blocking oligonucleotide 10 comprises, preferably consists of, the sequences:
third blocking oligonucleotide 10C: 5′-TTTTTTTGCYGCCCACTCAGACTTTA-3′ (SEQ ID NO: 82);
fourth blocking oligonucleotide 10D: 5′-TTTTTG+CYGCCC+ACTCAG+ACTTTA+TTC-3′ (SEQ ID NO: 83), wherein +C, +A and +T denote LNA nucleotides 44;
fifth blocking oligonucleotide: 5′-TTTTTTTTTTTTGCYGCCCACTCAGACTTTATTCAAAGAC-3′ (SEQ ID NO: 4);
second blocking oligonucleotide 10B: 5′-TTTTTTTTTTTTTTTGCYGCCCACTCAGACTTTATTCA AAGACCA-3′ (SEQ ID NO: 84);
first blocking oligonucleotide 10A: 5′-CGCGAGCGCGCCAGGGTTTATG(Xₙ)_{N}TTTTTTTTTTTTTT TGCYGCCCACTCAGACTTTA TTCAAAGAC-3′; wherein Xₙ is A, T, G or C, n=1-N and N is from 40 to 60;
first blocking oligonucleotide 10A: 5′-CGCGAGCGCGCCAGGGTTTATGTAATTAGAATTAGAAT GAATAGCTAACCTGATATGTTGAAGAACTATGACAGACATTTTTTTTTTTTTTTGCYGCCCACTCAG ACTTTATTCAAAGAC-3′ (SEQ ID NO: 8); and
variants of these sequences in which a nucleotide ata 3′-end 14 is phosphorylated 40 or is a ZNA nucleotide 42.

In another embodiment, the blocking oligonucleotide comprises, preferably consists of, the sequence of 5′-AGTGAACACAGTTGTGTCAGAAGCAAATGT(Xₘ)_{M}TTTTTTTTTTTTTTTGCAATGAAAATAAATGTT TTTTATTAGGCAG-3′, wherein Xₘ is A, T, G or C, m=1-M and M is from 40 to 60. In a preferred embodiment, the blocking oligonucleotide comprises, preferably consists of, the sequence of 5′-AGTGAACACAGTTGTGTCAGAAGCAAATGTAGAATGAATAGCTAACCTGATATGTTGAAGAACTAT GACAGACCTTTTTTTTTTTTTTTGCAATGAAAATAAATGTTTTTTATTAGGCAG-3′ (SEQ ID NO: 10), and more preferably 5′-AGTGAACACAGTTGTGTCAGAAGCAAATGTAGAATGAATAGCTAACCTGA TATGTTGAAGAACTATGACAGACCTTTTTTTTTTTTTTTGCAATGAAAATAAATGTTTTTTATTAGGCA G-Pho-3′ (SEQ ID NO: 11), wherein Pho denotes phosphorylation. Such blocking oligonucleotides are designed to bind to globin β mRNA molecules.

Further preferred embodiments of the blocking oligonucleotide 10 comprises, preferably consists of, the sequences:
third blocking oligonucleotide 10C: 5′-TTTTTTTTTTTTGCAATGAAAATAAATGTTTTTTATTAGG-3′ (SEQ ID NO: 85);
fourth blocking oligonucleotide 10D: 5′-TTTTTTTTTTG+CAATGA+AAATAA+ATGTTT+TTTAT TAGG-3′ (SEQ ID NO: 86), wherein +C, +A and +T denote LNA nucleotides 44;
second blocking oligonucleotide 10B: 5′-TTTTTTTTTTTTTTTGCAATGAAAATAAATGTTTTTTA TTAGGCAGAATCCAGAT-3′ (SEQ ID NO: 87);
fifth blocking oligonucleotide 10E: 5′-TTTTTTTTTTTTTTTGCAATGAAAATAAATGTTTTTTATTA GGCAG-3′ (SEQ ID NO: 5);
first blocking oligonucleotide: 5′-AGTGAACACAGTTGTGTCAGAAGCAAATGT(Xₘ)_{M}TTTTTTT TTTTTTTTGCAATGAAAATA AATGTTTTTTATTAGGCAG-3′, wherein Xₘ is A, T, G or C, m=1-M and M is from 40 to 60;
first blocking oligonucleotide 10A: 5′-AGTGAACACAGTTGTGTCAGAAGCAAATGTAGAATGAA TAGCTAACCTGATATGTTGAAGAACTATGACAGACCTTTTTTTTTTTTTTTGCAATGAAAATAAATGT TTTTTATTAGGCAG-3′ (SEQ ID NO: 10); and
variants of these sequences in which a nucleotide at a 3′-end 14 is phosphorylated 40 or is a Zip nucleotide 42.

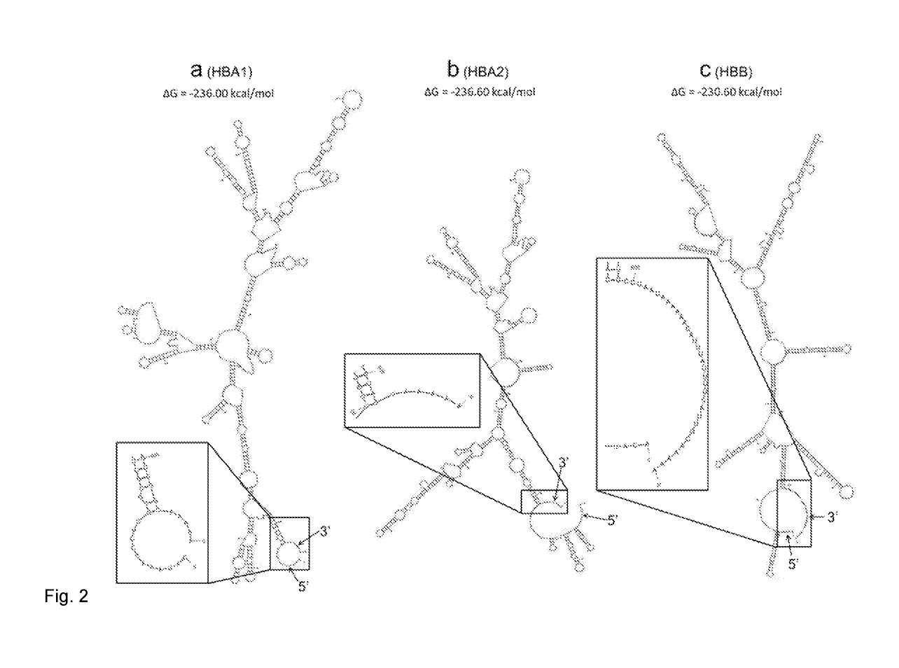

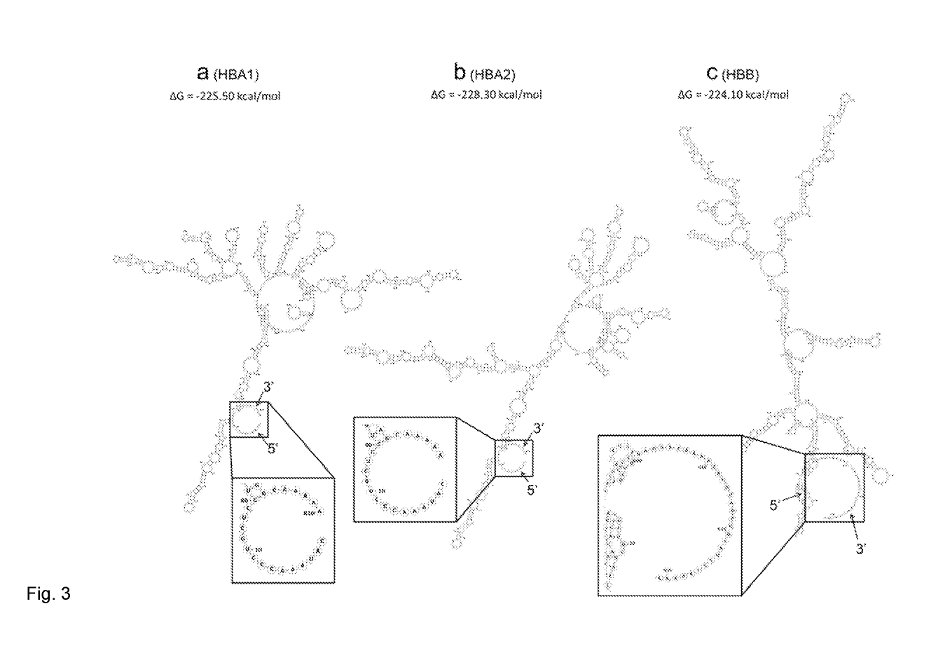

FIGS. 2A to 2C and 3A to 3C illustrate secondary structure of globin mRNA HBA1, HBA2 and HBB predicted by mFold software [7] and Vienna RNA software [8], respectively. The 3′ and 5′ ends are zoomed in to demonstrate the close proximity of both ends. The prediction demonstrates that the 3′ end of HBA1 and HBA2 are tightly packed to the secondary structure, leaving the binding region of blocking oligonucleotides masked even after temperature mediated globin mRNA denaturation. The HBB 3′ UTR has open structure, enabling efficient hybridization of the blocking oligonucleotides.

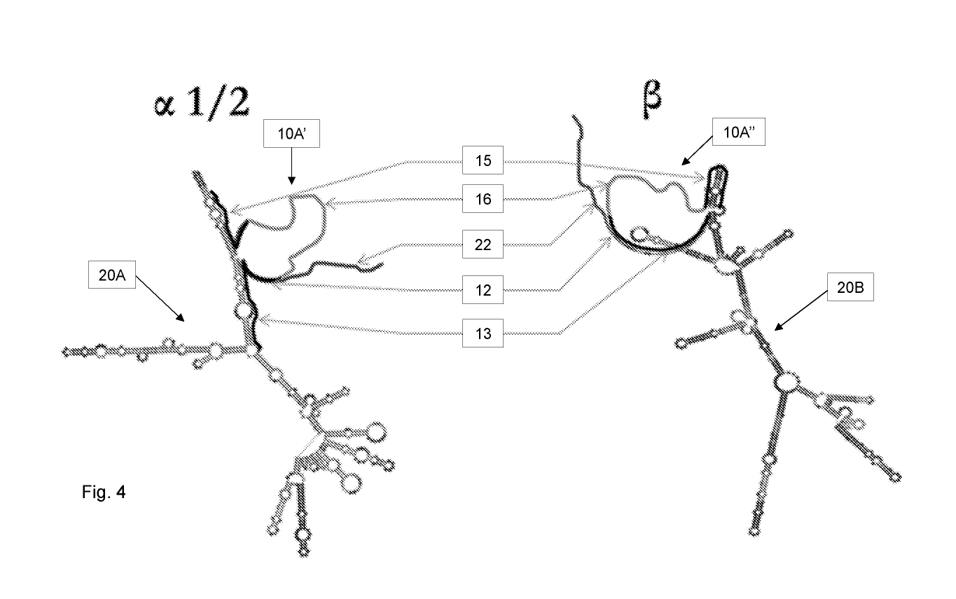

FIG. 4 illustrates the secondary structure of a globin α1/2 mRNA molecule 20A and a globin β mRNA molecule 20B and binding of blocking oligonucleotides 10A′, 10A″ according to the first blocking oligonucleotide embodiments to such globin α1/2 and β mRNA molecules 20A, 20B. The figure shows the 5′-end complementary sequence 15, the linker sequence 16, the poly-A complementary sequence 12 and the 3′-end complementary sequence 13 of the blocking oligonucleotides 10A′, 10A″. The figure also indicates the poly-A tails 22 of the globin mRNA molecules 20A, 20B.

Another aspect of the embodiments relates to a double strand complex comprising a globin mRNA molecule and a blocking oligonucleotide according to the embodiments hybridized to at least a portion of the globin mRNA molecule. FIGS. 1 and 4 illustrate such double strand complexes.

A further aspect of the embodiments relates to a sample comprising at least one RNA molecule and a double strand complex according to above.

The sample is preferably a biological sample comprising RNA molecules, and more preferably a body sample or biopsy from an animal, preferably from a mammal and more preferably from a human. In a particular embodiment, the sample is a type of liquid biopsy and preferably a blood sample or a serum sample comprising RNA molecules.

At least one type of blocking oligonucleotides according to the embodiments has been added to the sample to bind to matching globin mRNA molecules present in the sample. This means that globin mRNA molecules in the sample form double strand complexes with the added blocking oligonucleotides. The blocking oligonucleotides, however, do not hybridize to non-globin mRNA molecules in the samples. Hence, such other RNA molecules (non-globin mRNA molecules) are present in the sample in their natural form unaffected by the presence of blocking oligonucleotides.

The sample preferably comprises both blocking oligonucleotides capable of forming double strand complexes with globin α mRNA molecules and blocking oligonucleotides capable of forming double strand complexes with globin β mRNA molecules.

The sample as defined above is thereby suitable for usage in a gene expression analysis involving addition of reverse transcription enzymes or an analogous enzyme to form cDNA molecules of the RNA molecules in the sample. In such a case, the blocking oligonucleotides prevent or at least significantly inhibit reverse transcription of any globin mRNA molecules in the sample by forming double strand complexes with the globin mRNA molecules and thereby preventing or at least significantly inhibiting reverse transcription anchored poly-T primers from binding to the globin mRNA molecules.

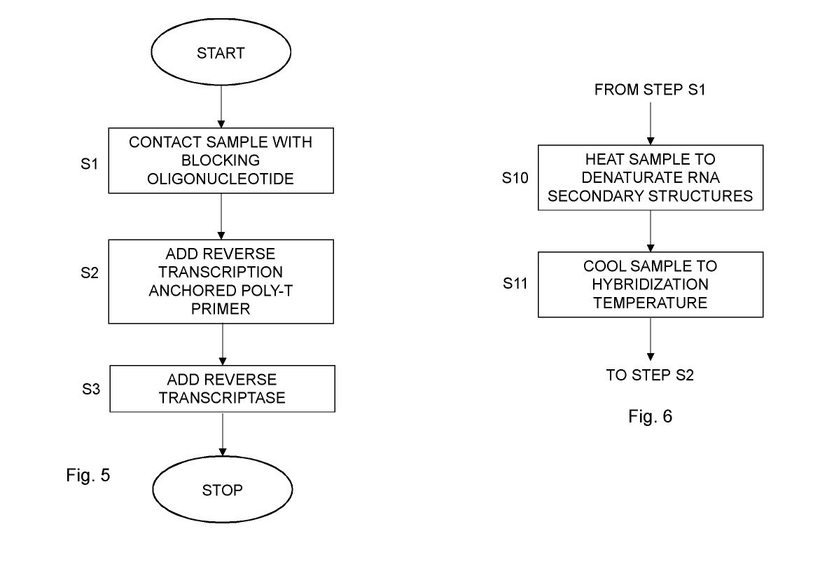

FIG. 5 is a flow chart illustrating a method of producing a cDNA molecule. The method comprising contacting, in step S1, a sample comprising at least one mRNA molecule and at least one globin mRNA molecule with at least one blocking oligonucleotide according to the embodiments under conditions enabling hybridization of a blocking oligonucleotide of the at least one blocking oligonucleotide to a globin mRNA molecule of the at least one globin mRNA molecule. The following steps comprise adding, in step S2, a reverse transcription anchored poly-T primer to the sample and adding, in step S3, a reverse transcription enzyme to the sample to produce the cDNA molecule from the at least one mRNA molecule.

The two steps S2 and S3 can be performed serially in any order or at least partly parallel. It is also possible to combine these two steps by adding a mixture comprising the reverse transcription anchored poly-T primer and the reverse transcription enzyme to the sample.

The reverse transcription enzyme added in step S3 or together with the reverse transcription anchored poly-T primer can be any enzyme capable of conducting reverse transcription and producing cDNA molecules from an mRNA template starting from a reverse transcription anchored poly-T primer. Non-limiting examples of such reverse transcription enzymes include reverse transcriptase and RNA-dependent polymerases.

In an embodiment, step S1 comprises contacting a blood sample comprising the at least one mRNA molecule and the at least one globin mRNA molecule with the at least one blocking oligonucleotide.

Hence, a blood sample, and in particular a whole blood RNA sample, is a preferred example of an mRNA-containing sample that can be in the method as shown in FIG. 5.

In an embodiment, step S1 comprises contacting the sample with at least one blocking oligonucleotide capable of hybridizing to globin α mRNA molecules and at least one blocking oligonucleotide capable of hybridizing to globin β mRNA molecules. In such a case, the blocking oligonucleotides will thereby prevent or at least significantly reduce reverse transcription of both globin α and β mRNA molecules present in the sample.

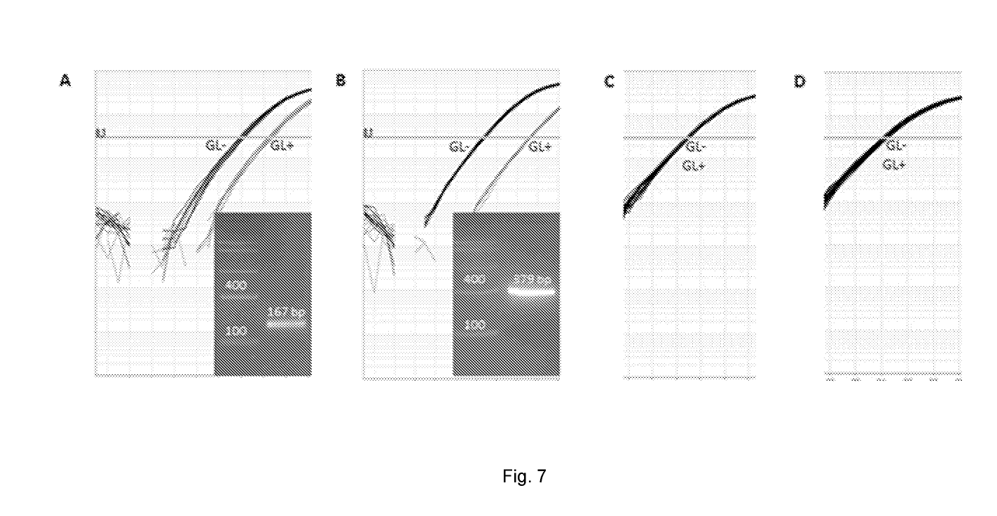

FIG. 6 is a flow chart illustrating additional, optional steps of the method shown in FIG. 5. The method continues from step S1 in FIG. 5. A next step S10 comprises heating the sample to denature RNA secondary structures following step S1 but prior to step S3. The method continues to step S11, which comprises cooling the sample from step S10 to a hybridization temperature for the at least one blocking oligonucleotide prior to step S3. The method then continues to step S2 in FIG. 5.

The heating of the sample in step S10 denature any RNA secondary structures thereby enabling the blocking oligonucleotides to access the relevant sequences (3′-end sequence and preferably 5-end sequence and poly-A sequence) of the globin mRNA molecules. The sample is then cooled down to at least below the hybridization temperature of the blocking oligonucleotides to allow them to hybridize to the globin mRNA molecules in the sample.

The hybridization temperature of the blocking oligonucleotide depends on the length of the respective complementary sequences of the blocking oligonucleotides and the degree of stringent annealing between the complementary sequences and the corresponding sequences of the globin mRNA molecules. Non-limiting examples of suitable hybridization temperature includes from about 30° C. to about 90° C., such as from 40° C. to 80° C., preferably from 50° C. to 70° C., and more preferably from 55° C. to 65° C.

A further aspect of the embodiments relates to a kit for producing a complementary cDNA molecule. The kit comprises at least one blocking oligonucleotide according to the embodiments and at least one reverse transcription anchored poly-T primer.

In an embodiment, the kit also comprises a reverse transcription enzyme.

The kit can be used in the method as described above in connection with FIGS. 5 and 6.

The invention allows applying the maximum power of RNA-seq to detect mRNAs at 1-100 ng range in wbRNA samples ignoring concurrently abundant globin mRNA molecules. The effect is achieved by highly specific globin mRNA molecular non-enzymatic manipulation that masks globin mRNA molecules for reverse transcription anchored poly-T primer priming prior to reverse transcription, thereby significantly reducing globin cDNA synthesis. As a result, globin mRNA is not available for reverse transcription, saving poly-T primers, nucleotides and enzyme activity for RNA molecules of interest.

The blocking oligonucleotides of the embodiments have been tested by two independent methods:
(i) globin α1/2 and β specific quantitative polymerase chain reaction (qPCR); and
(ii) massively parallel RNA-seq.

The qPCR-based assay confirmed globin α1/2 cDNA synthesis reduction by 8.8 times and globin β cDNA synthesis reduction by more than 10 times, respectively. RNA-seq detected very low expression levels of globin β, having 0.5% prevalence among all expressed genes. Globin α1/2, with a prevalence of 4.7%, had somehow higher expression level but was still a dimension lower than would be expected without treatment (21.4%) of the sample with the blocking oligonucleotides of the embodiments. Artificial RNA spikes were used to confirm high specificity of the blocking oligonucleotides. The blocking oligonucleotides had no negative effect to overall RNA quality, providing as high as 80-85% endogenous 5′-end capture rate and 89-91% RNA spike 5′-end capture rate.

As shown in the experimental section, extracted total blood-RNA is added to GL buffer A (GL-K⁺) to form stable complexes between the blocking oligonucleotides and globin mRNA molecules during specific hybridization. GL buffer B is then added to initiate cDNA synthesis. The characteristics of the blocking oligonucleotides enable incorporation of the blocking oligonucleotides already in the RNA denaturation buffer, thereby requiring no additional hands-on steps. The only addition to the protocol of standard cDNA synthesis is to allow the blocking oligonucleotides to hybridize to the globin mRNA molecules (hybridization time about 10 min). No RNA degradation was detected after qPCR- or RNA-seq procedures.

Thus, the hybridization of the blocking oligonucleotides to globin mRNA molecules takes place prior to cDNA synthesis. For instance, extracted wbRNA (commonly 1-100 ng or higher) is added to GL buffer A that comprises proper hybridization buffer and blocking oligonucleotides. The wbRNA sample is heated once to denature RNA secondary structure (commonly at 95° C.) and then cooled to the hybridization temperature of the blocking oligonucleotides (commonly at 55-65° C.) for 10 min, for example, to enable formation of double strand (circular) complexes between blocking oligonucleotides and globin mRNA molecules. The sample is then cooled to about 42° C. on cycler to add GL buffer B to initiate cDNA synthesis at 42° C. (commonly 30-90 min) until cDNA is generated and the reverse transcription enzyme is inactivated for further manipulations.

In a particular embodiment, at least one blocking oligonucleotide is used to mask globin α1/2 mRNA and at least one blocking oligonucleotide is used to mask globin β mRNA in order to inhibit globin cDNA synthesis, which is mediated by reverse transcription anchored poly-T primers. As a result, globin mRNA molecules are not available for reverse transcription, thereby saving poly-T primers, nucleotides and enzyme activity for RNA molecules of interest.

The blocking oligonucleotides use synthetic DNA, RNA or analog oligonucleotide mediated nucleic acid hybridization to mask globin α1/2 mRNA and globin β mRNA. This masking reaction takes place prior to cDNA synthesis. The blocking oligonucleotides are designed so that they have at least one highly complementary region to the globin mRNA molecule—the 3′-end complementary sequence 13 or the combined sequence 11 with the 3′-end complementary sequence 13 and the poly-A complementary sequence 12 in FIG. 1. In particular, the combined sequence 11 is highly complementary to the up- and downstream mRNA sequence of the poly-A tail 5′ start site of globin α1/2 and β mRNA molecules 20. In other words, the blocking oligonucleotides 10 bind specifically to the gene coding 23 and poly-A tail 22 joint region of the globin mRNA 3′ region. The function of the blocking oligonucleotides 10 is to mask the anchored poly-T primer priming site of globin mRNA molecules 20 to thereby significantly decrease globin cDNA synthesis.

The gene coding and poly-A tail joint sequence of globin α1 mRNA is the following:

(SEQ ID NO: 12)
5′ . . . UGGUCUUUGAAUAAAGUCUGAGUGGGCGGCAAAAAAAA
AA . . . 3′.

The complementary region of a blocking oligonucleotide that is capable of hybridizing to globin α1 mRNA molecules is 3′ . . . ACCAGAAACTTATTTCAGACTCACCCGCCGTTTTTTTTTT . . . 5′ (SEQ ID NO: 13).

The gene coding and poly-A tail joint sequence of globin α2 mRNA is the following:

(SEQ ID NO: 14)
5′ . . . UGGUCUUUGAAUAAAGUCUGAGUGGGCAGCAAAAAAAA
AA . . . 3′.

The complementary region of a blocking oligonucleotide that is capable of hybridizing to globin α2 mRNA molecules is 3′ . . . ACCAGAAACTTATTTCAGACTCACCCGTCGTTTTTTTTTT . . . 5′ (SEQ ID NO: 15).

Previous underlined nucleotides represent sequence variability between globin α1 and α2 mRNA at the coding 3′-end. Accordingly, a corresponding blocking oligonucleotide that is complementary to both globin α1/2 mRNA can be 3′ . . . ACCAGAAACTTATTTCAGACTCACCCGYCGTTTTTTTTTT . . . 5′ (SEQ ID NO: 16), wherein Y means T or C nucleotide.

The gene coding and poly-A tail joint sequence of globin β mRNA is the following:

(SEQ ID NO: 17)
5′ . . . CUGCCUAAUAAAAAACAUUUAUUUUCAUUGCAAAAAAA
AAA . . . 3′.

The complementary region of a blocking oligonucleotide that is capable of hybridizing to globin β mRNA molecules is 3′ . . . GACGGATTATTTTTTGTAAATAAAAGTAACGTTTTTTTTTT . . . 5′ (SEQ ID NO: 18).

The 5′-end complementary sequence 15 of the blocking oligonucleotides 10 is highly complementary to the globin mRNA 5′-end sequence 25. The functions of the 5′-end complementary sequence 15 are (i) to increase specificity of the blocking oligonucleotides 10 to globin mRNA molecules 20 and (ii) to enable globin mRNA circular locking prior to cDNA synthesis.

The linker sequence 16 of the blocking oligonucleotides has linker function between the combined sequence 11 and the 5′-end complementary sequence 15.

Due to the nature of stable globin mRNA secondary and tertiary structure, globin mRNA 3-′ and 5′-ends are located physically close to each other as shown in FIG. 2. This provides an opportunity to use blocking oligonucleotides 10A, 10B to join both ends of the globin mRNA molecules 20A, 20B to form closed, highly specific, and stable complexes between blocking oligonucleotides 10A, 10B and globin mRNA molecules 20A, 20B as shown in FIG. 2.

The blocking oligonucleotides 10A, 10B have at least one specific region 12, 13 but preferably two specific regions 12, 13, 15 capable of hybridizing specifically to globin mRNA molecules 20A, 20B. A blocking oligonucleotide with one specific region 12, 13 has 20% lower globin α1/2 mRNA depletion effect as compared to a blocking oligonucleotide with two specific regions 12, 13, 15. No significant difference was, however, detected for globin β mRNA reduction when blocking oligonucleotides with one specific region 12, 13 or two specific regions 12, 13, 15 were compared.

The 5′-end complementary sequence, preferably 15-35 nt, bind to the 5′-end sequence of globin mRNA and covers the mRNA CAP structure. The combined sequence preferably comprises the 3′-end complementary sequence, preferably 20-40 nt, complementary to the 3′-end sequence of globin mRNA and the poly-A complementary sequence, preferably 1-30 nt, complementary to the poly-A tail of globin mRNA. This combined sequence thereby achieves a high specificity to the 3′-end of globin mRNA. The middle part of the blocking oligonucleotide is a linker sequence, preferably 40-60 nt, having linker function to ensure enough mobility between the two specific regions of the blocking oligonucleotide. The linker sequence also provides connecting function in case of globin α1/2 and β mRNA locking. The 3′-end of the blocking oligonucleotide is preferably chemically blocked, such as phosphorylated, to avoid extension of the blocking oligonucleotide during reverse transcription as well as further possible enzymatic extensions.

An example of a universal blocking oligonucleotide for human globin α1/2 mRNA is:

(SEQ ID NO: 9)
5′- [symbol] TAATTAGAATTAGAATGA
ATAGCTAACCTGAYATGTTGAAGAACTATGACAGACATTTTTTT
TTTTTTTTGCYGCCCACTCAGACTTTATTCAAAGAC-Pho-3′.

Strikethrough sequence is complementary to the 5′-end sequence of globin α1/2 mRNA. The underlined sequence hybridizes to the 3′ coding sequence of the globin α1/2 mRNA and the 5′-end of the poly-A tail of the globin α1/2 mRNA. The intermediate sequence has linker function, having no sequence preferences. The 3′-end of the blocking oligonucleotide is chemically blocked by phosphate, for example, to eliminate the risk of extension during cDNA synthesis or further cDNA amplification.

Human globin α1/2 full mRNA sequences are depicted below. The sequences are in 5′-3′ orientation and the sequences to which the blocking oligonucleotide binds are marked by strikethrough and underlining. Nucleotide difference between globin α1/2 mRNA at the 3′-region is marked as bold. The universal blocking oligonucleotide contains a Y nucleotide variant (C/T) to be universal for both globin α1/2 globin mRNAs.

>gi|672228742|ref|NM_000558.4| Homo sapiens
hemoglobin, alpha 1 (HBA1), mRNA
(SEQ ID NO: 19) [symbol]
GCCCGGCACUCUUCUGGUCCCCAC
AGACUCAGAGAGAACCCACCAUGGUGCUGUCUCCUGCCGACAAGACC
AACGUCAAGGCCGCCUGGGGUAAGGUCGGCGCGCACGCUGGCGAGUA
UGGUGCGGAGGCCCUGGAGAGGAUGUUCCUGUCCUUCCCCACCACCA
AGACCUACUUCCCGCACUUCGACCUGAGCCACGGCUCUGCCCAGGUU
AAGGGCCACGGCAAGAAGGUGGCCGACGCGCUGACCAACGCCGUGGC
GCACGUGGACGACAUGCCCAACGCGCUGUCCGCCCUGAGCGACCUGC
ACGCGCACAAGCUUCGGGUGGACCCGGUCAACUUCAAGCUCCUAAGC
CACUGCCUGCUGGUGACCCUGGCCGCCCACCUCCCCGCCGAGUUCAC
CCCUGCGGUGCACGCCUCCCUGGACAAGUUCCUGGCUUCUGUGAGCA
CCGUGCUGACCUCCAAAUACCGUUAAGCUGGAGCCUCGGUGGCCAUG
CUUCUUGCCCCUUGGGCCUCCCCCCAGCCCCUCCUCCCCUUCCUGCA
CCCGUACCCCCGUGGUCUUUGAAUAAAGUCUGAGUGGGCGGCAAAAA
AAAAAAAAAAAAA . . .
>gi|172072689|ref|NM_000517.4| Homo sapiens
hemoglobin, alpha 2 (HBA2), mRNA
(SEQ ID NO: 20) [symbol]
GGCCGGCACUCUUCUGGUCCCCAC
AGACUCAGAGAGAACCCACCAUGGUGCUGUCUCCUGCCGACAAGACC
AACGUCAAGGCCGCCUGGGGUAAGGUCGGCGCGCACGCUGGCGAGUA
UGGUGCGGAGGCCCUGGAGAGGAUGUUCCUGUCCUUCCCCACCACCA
AGACCUACUUCCCGCACUUCGACCUGAGCCACGGCUCUGCCCAGGUU
AAGGGCCACGGCAAGAAGGUGGCCGACGCGCUGACCAACGCCGUGGC
GCACGUGGACGACAUGCCCAACGCGCUGUCCGCCCUGAGCGACCUGC
ACGCGCACAAGCUUCGGGUGGACCCGGUCAACUUCAAGCUCCUAAGC
CACUGCCUGCUGGUGACCCUGGCCGCCCACCUCCCCGCCGAGUUCAC
CCCUGCGGUGCACGCCUCCCUGGACAAGUUCCUGGCUUCUGUGAGCA
CCGUGCUGACCUCCAAAUACCGUUAAGCUGGAGCCUCGGUAGCCGUU
CCUCCUGCCCGCUGGGCCUCCCAACGGGCCCUCCUCCCCUCCUUGCA
CCGGCCCUUCCUGGUCUUUGAAUAAAGUCUGAGUGGGCAGCAAAAAA
AAAAAAAAAAAA . . .

An example of a blocking oligonucleotide for human globin β mRNA is shown below:

(SEQ ID NO: 10)
5′- [symbol] AGAATGAATAGCT
AACCTGATATGTTGAAGAACTATGACAGACCTTTTTTTTTTTTTTTG
CAATGAAAATAAATGTTTTTTATTAGGCAG-Pho-3′.

The strikethrough sequence represents the 5′-end complementary sequence that hybridizes to the 5′-end sequence of globin β mRNA molecules. The linker sequence has no sequence preferences. The underlined poly-T part hybridizes to the poly-A tail of the globin β mRNA, blocking the priming site of reverse transcription anchored poly-T primers. The rest of the underlined sequence represents the 3′-end complementary sequence that hybridizes to the 3′-end sequence of globin β mRNA. The 3′-end of the blocking oligonucleotide is chemically blocked by phosphate, for example, to eliminate the risk of extension during cDNA synthesis or further cDNA amplification.

Human globin β full mRNA sequence is depicted below. The sequences have 5′-3′ orientation and the sequences to which the blocking oligonucleotide binds are marked by strikethrough and underlining.

>gi|28302128|ref|NM_000518.4| Homo sapiens
hemoglobin, beta (HBB), mRNA
(SEQ ID NO: 21) [symbol]
AGCAACCUCAAACAGA
CACCAUGGUGCAUCUGACUCCUGAGGAGAAGUCUGCCGUUACUGCCC
UGUGGGGCAAGGUGAACGUGGAUGAAGUUGGUGGUGAGGCCCUGGGC
AGGCUGCUGGUGGUCUACCCUUGGACCCAGAGGUUCUUUGAGUCCUU
UGGGGAUCUGUCCACUCCUGAUGCUGUUAUGGGCAACCCUAAGGUGA
AGGCUCAUGGCAAGAAAGUGCUCGGUGCCUUUAGUGAUGGCCUGGCU
CACCUGGACAACCUCAAGGGCACCUUUGCCACACUGAGUGAGCUGCA
CUGUGACAAGCUGCACGUGGAUCCUGAGAACUUCAGGCUCCUGGGCA
ACGUGCUGGUCUGUGUGCUGGCCCAUCACUUUGGCAAAGAAUUCACC
CCACCAGUGCAGGCUGCCUAUCAGAAAGUGGUGGCUGGUGUGGCUAA
UGCCCUGGCCCACAAGUAUCACUAAGCUCGCUUUCUUGCUGUCCAAU
UUCUAUUAAAGGUUCCUUUGUUCCCUAAGUCCAACUACUAAACUGGG
GGAUAUUAUGAAGGGCCUUGAGCAUCUGGAUUCUGCCUAAUAAAAAA
CAUUUAUUUUCAUUGCAAAAAAAAAAAAAAAAAA . . .

An example of a reverse transcription anchored poly-T primer is 5′-TTTTTTTTTTTTTTTTTTTTTTTTTVN-3′ (SEQ ID NO: 1). The reverse transcription anchored poly-T primer is preferably 15-30 nt long. The 3′-end of the reverse transcription anchored poly-T primer preferably contains 3′-NV-5′, wherein ‘N’ is a mix of all four bases and ‘V’ is a mix of ‘A’ or ‘C’ or ‘G’. This VN sequence directs the reverse transcription anchored poly-T primer to the beginning (5′-end) of the mRNA poly-A tail. The 3′-end of the reverse transcription anchored poly-T primer could contain only the “V” nucleotide as well to mark the beginning of coding mRNA region but “NV” adds more specificity.

### EXAMPLES

In the examples, the blocking oligonucleotides of the embodiments are denoted GlobinLock (GL).

The following blocking oligonucleotides and artificial RNA spike-in molecules were used in Example 1-3:

GL α1/2 (SEQ ID NO: 9):
5′-CGCGAGCGCGCCAGGGTTTATGTAATTAGAATTAGAATGAATAG
CTAACCTGATATGTTGAAGAACTATGACAGACATTTTTTTTTTTTTT
TGCYGCCCACTCAGACTTTATTCAAAGAC-Pho-3′
Y = C for GL α1 and Y = T for GL α2
GL β (SEQ ID NO: 11):
5′-AGTGAACACAGTTGTGTCAGAAGCAAATGTAGAATGAATAGCTA
ACCTGATATGTTGAAGAACTATGACAGACCTTTTTTTTTTTTTTTGC
AATGAAAATAAATGTTTTTTATTAGGCAG-Pho-3′
Reverse transcription anchored poly-T primer
with universal linker (SEQ ID NO: 22):
5′-amine-GTGATCTTGAACAGAGTGTCAGTGCGATTTTTTTTTTT
TTTTTTTTTTTTTTTTTTVN-3′,
wherein V is a mix of A, C and G and N is a mix
of A, T, U, C and G.
qPCR globin α1/2 detection primer left
(SEQ ID NO: 23):
ACTCTTCTGGTCCCCACAGACTC
qPCR globin α1/2 detection primer right
(SEQ ID NO: 24):
AGTAGGTCTTGGTGGTGGGGAA
qPCR globin β detection primer left
(SEQ ID NO: 25):
CTTTGCCACACTGAGTGAGCTG
qPCR globin β detection primer right
(SEQ ID NO: 26):
GTGATCTTGAACAGAGTGTCAGTGCTGA
pSPIKE1 (RNA_SPIKE_1_EC2) left primer
(SEQ ID NO: 27) and right primer
(SEQ ID NO: 28):
Left: CTGCATTGACGGTGCTGACC
Right: CGGCATCGCATTGCCCATAA
pSPIKE2 (RNA_SPIKE_2_EC12) left primer
(SEQ ID NO: 29) and right primer
(SEQ ID NO: 30):
Left: AAGGTTCCCGTGCCAGTTGA
Right: GCTTCTGAAAACCGCCTCGG

Artificial spike-in molecules from Ambion (Cat no AM1780) were used. RNA Spike 1 (750 bp) and RNA Spike 2 (752 bp) were diluted and added to total blood-RNA before GL reaction and cDNA synthesis to measure specificity of GL.

### Example 1

### Globin Reduction Quantitative Assay

Globin cDNA synthesis reduction was tested by qPCR that was specific to cDNA created from globin α1/2 mRNA as well as globin β mRNA. 1 μL of total blood-RNA (50 ng/μL, including spike-in controls) was added to 5 μl GL mixture A (GL-K⁺) that contained 1 M betaine (Sigma Aldrich), 20% PEG-4000 (Sigma), 2 mM dNTP mixture (Thermo), 10 mM Tris-HCl (pH 8.0, Sigma), 150 mM KCl (Sigma), 0.2% Triton X-100 (Sigma), and 5 μM GL α1/2 oligonucleotide and 2 μM of GL β oligonucleotide (both from Sigma). Total wbRNA was denatured 1 min at 95° C. and incubated 10 min at 60° C. for GL hybridization. After 10 minutes the GL-treated RNA sample was cooled to 42° C. Five μl of GL mixture B was added to initiate cDNA synthesis. The 5 μl GL B mixture contained 1 M betaine, 100 mM Tris-HCl (pH 8.0), 10 mM DTT (Sigma), 15 mM MgCl₂ (Sigma), 7 U RiboLOCK (Thermo), 800 nM poly-T anchored primer with universal linker (Sigma), and 70 U RevertAid Premium reverse transcriptase (Thermo). Samples were incubated 60 min at 42° C. and the reverse transcription enzyme was inactivated by 5 min at 85° C. Globin synthesis yield was quantified directly by qPCR using GL positive and negative samples and quantitative 10× dilutions from cDNA synthesis. For qPCR, 1 μl (10% of reaction outcome) of undiluted synthesized cDNA was used as template and 19 μl of qPCR mixture was added. The qPCR mixture contained 4 μl of 5× Solis SYBR ROX mix (SolisBiodyne) and globin α1/2 and β qPCR detection primers at 200 nM final concentration. ABI7500 Fast instrument was used to carry out qPCR-based globin cDNA validation. Regular ramp speed was used. Initial enzyme activation was at 15 min at 95° C., thereupon 15 s at 95° C., 20 s at 62° C., and 30 s at 72° C. (primer extension and SYBR detection time) cycle was used 25 times.

Ambion Array Control RNA Spike-1 and Spike-2 artificial RNAs were used to evaluate GL specificity and GL overall impact on reverse transcription. RNA Spike 1 and Spike 2 mixture was added together with blood total-RNA to GL+ and GL− master mix during GL reaction. After cDNA synthesis, spike cDNA yield was quantified by qPCR simultaneously with GL α1/2 and β reduction.

qPCR amplicon sequence of globin α1 cDNA
(SEQ ID NO: 31):
CATAAACCCTGGCGCGCTCGCGGCCCGGCACTCTTCTGGTCCCCACA
GACTCAGAGAGAACCCACCATGGTGCTGTCTCCTGCCGACAAGACCA
ACGTCAAGGCCGCCTGGGGTAAGGTCGGCGCGCACGCTGGCGAGTAT
GGTGCGGAGGCCCTGGAGAGGATGTTCCTGTCCTTCCCCACCACCAA
GACCTACTTCCCGCACTTCGACCTGAGCCACGGCTCTGCCCAGGTTA
AGGGCCACGGCAAGAAGGTGGCCGACGCGCTGACCAACGCCGTGGCG
CACGTGGACGACATGCCCAACGCGCTGTCCGCCCTGAGCGACCTGCA
CGCGCACAAGCTTCGGGTGGACCCGGTCAACTTCAAGCTCCTAAGCC
ACTGCCTGCTGGTGACCCTGGCCGCCCACCTCCCCGCCGAGTTCACC
CCTGCGGTGCACGCCTCCCTGGACAAGTTCCTGGCTTCTGTGAGCAC
CGTGCTGACCTCCAAATACCGTTAAGCTGGAGCCTCGGTGGCCATGC
TTCTTGCCCCTTGGGCCTCCCCCCAGCCCCTCCTCCCCTTCCTGCAC
CCGTACCCCCGTGGTCTTTGAATAAAGTCTGAGTGGGCGGCAAAAAA
AAAAAAAAAAAA . . .
qPCR amplicon sequence of globin α2 cDNA
(SEQ ID NO: 32):
CATAAACCCTGGCGCGCTCGCGGGCCGGCACTCTTCTGGTCCCCACA
GACTCAGAGAGAACCCACCATGGTGCTGTCTCCTGCCGACAAGACCA
ACGTCAAGGCCGCCTGGGGTAAGGTCGGCGCGCACGCTGGCGAGTAT
GGTGCGGAGGCCCTGGAGAGGATGTTCCTGTCCTTCCCCACCACCAA
GACCTACTTCCCGCACTTCGACCTGAGCCACGGCTCTGCCCAGGTTA
AGGGCCACGGCAAGAAGGTGGCCGACGCGCTGACCAACGCCGTGGCG
CACGTGGACGACATGCCCAACGCGCTGTCCGCCCTGAGCGACCTGCA
CGCGCACAAGCTTCGGGTGGACCCGGTCAACTTCAAGCTCCTAAGCC
ACTGCCTGCTGGTGACCCTGGCCGCCCACCTCCCCGCCGAGTTCACC
CCTGCGGTGCACGCCTCCCTGGACAAGTTCCTGGCTTCTGTGAGCAC
CGTGCTGACCTCCAAATACCGTTAAGCTGGAGCCTCGGTAGCCGTTC
CTCCTGCCCGCTGGGCCTCCCAACGGGCCCTCCTCCCCTCCTTGCAC
CGGCCCTTCCTGGTCTTTGAATAAAGTCTGAGTGGGCAGCAAAAAAA
AAAAAAAAAAA . . .

Primer binding regions are underlined and amplicons are shown as bold. Globin α1/2 qPCR product is 167 nt.

qPCR amplicon sequence of globin β cDNA
(SEQ ID NO: 33):
ACATTTGCTTCTGACACAACTGTGTTCACTAGCAACCTCAAACAGACA
CCATGGTGCATCTGACTCCTGAGGAGAAGTCTGCCGTTACTGCCCTGT
GGGGCAAGGTGAACGTGGATGAAGTTGGTGGTGAGGCCCTGGGCAGGC
TGCTGGTGGTCTACCCTTGGACCCAGAGGTTCTTTGAGTCCTTTGGGG
ATCTGTCCACTCCTGATGCTGTTATGGGCAACCCTAAGGTGAAGGCTC
ATGGCAAGAAAGTGCTCGGTGCCTTTAGTGATGGCCTGGCTCACCTGG
ACAACCTCAAGGGCACCTTTGCCACACTGAGTGAGCTGCACTGTGACA
AGCTGCACGTGGATCCTGAGAACTTCAGGCTCCTGGGCAACGTGCTGG
TCTGTGTGCTGGCCCATCACTTTGGCAAAGAATTCACCCCACCAGTGC
AGGCTGCCTATCAGAAAGTGGTGGCTGGTGTGGCTAATGCCCTGGCCC
ACAAGTATCACTAAGCTCGCTTTCTTGCTGTCCAATTTCTATTAAAGG
TTCCTTTGTTCCCTAAGTCCAACTACTAAACTGGGGGATATTATGAAG
GGCCTTGAGCATCTGGATTCTGCCTAATAAAAAACATTTATTTTCATT
GCAAAAAAAAAAAAAAATCAGCACTGACACTCTGTTCAAGATCAC

Primer binding regions are underlined and amplicons are shown as bold. Globin β qPCR product is 379 nt.

### Example 2

### GlobinLock Mediated Globin Reduction

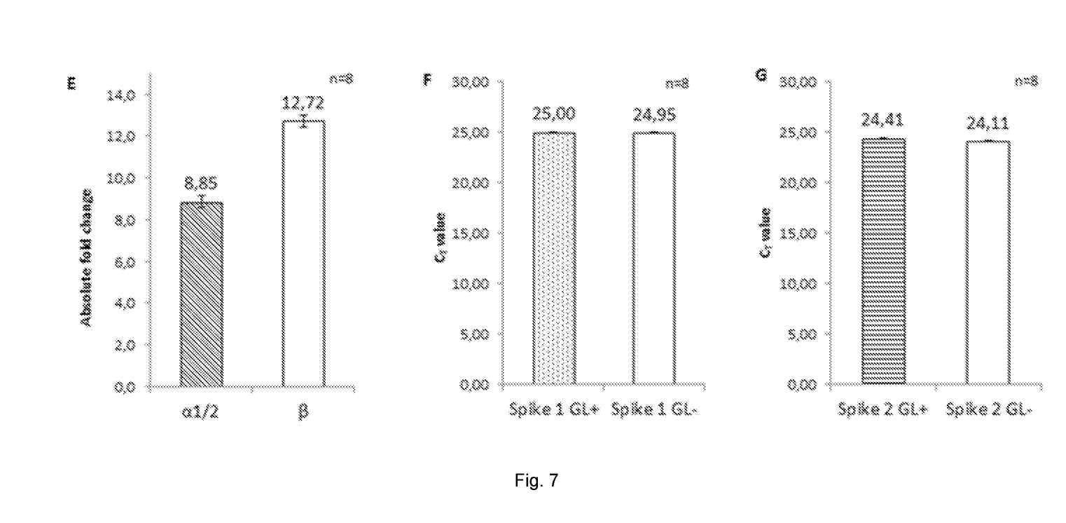

Globin α1/2 and β globin cDNA synthesis inhibition through GL action was quantified directly after blood total-RNA cDNA synthesis using qPCR assay. Globin reduction was first determined using qPCR delta cycle threshold (Ct) differences between GL positive and negative samples (n=8). As qPCR is a relative method without standards, 10× dilution of GL positive and negative samples (n=8) was used to determine delta Ct value which indicated ten time input reduction and converted Ct values to absolute fold change values. One universal qPCR primer pair was designed to determine globin α1/2 cDNA molecules. Globin R cDNA molecule was determined by another primer pair in a separate reaction tube using GL positive (n=8) and negative (n=8) samples. Globin α1/2 cDNA synthesis was reduced 8.8±0.3× using cDNA as template. Globin β cDNA reduction was >10× (12.7±0.3×), see FIGS. 7A, 7B and 7E.

GL specificity was measured by two artificial RNA spike-in molecules. Spike 1 Ct value in GL+ was 25.00±0.02 and in GL− 24.95±0.05. Spike 2 Ct value in GL+ was 24.41±0.03 and in GL− 24.11±0.04, see FIGS. 7C, 7D, 7F and 7G. Based on the two spike-in references, GL has high specificity to target globin molecules and has no negative effect on reverse transcription reaction as well as no poly-A-based specificity to the rest of the poly-A tailed mRNAs.

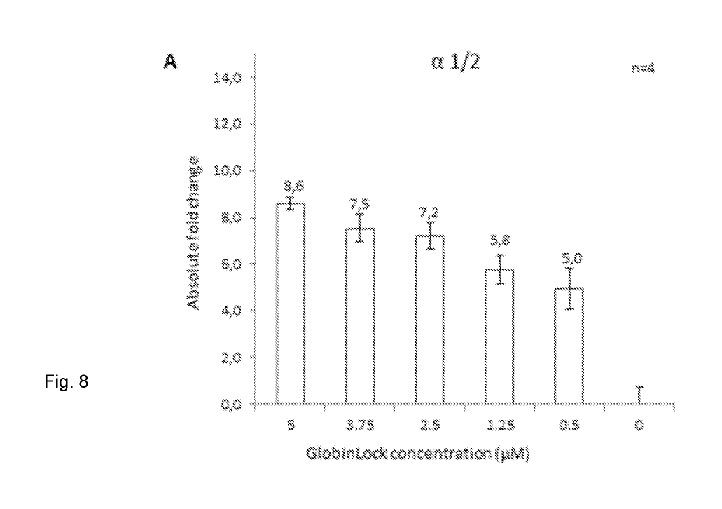

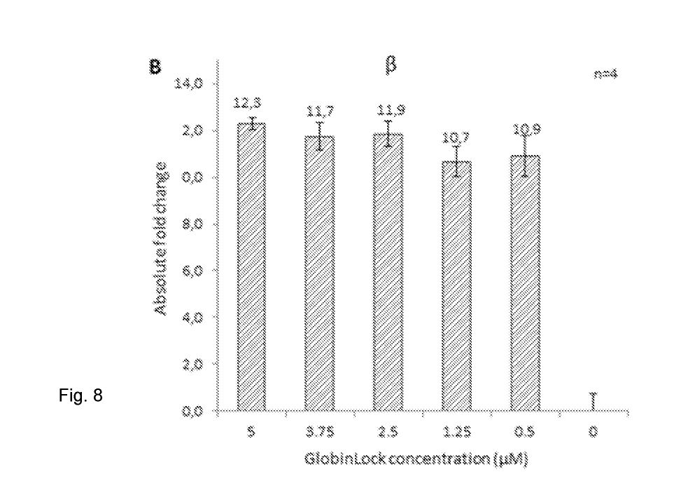

GL oligonucleotide concentration was titrated using different GL concentrations from 0.5 μM to 5 μM following previously described protocol. Highest GL sensitivity was achieved at 5 μM concentration in both α1/2 and β gmRNAs. GL α1/2 are sensitive to GL concentration having 40% blocking decrease in case of 10× GL concentration reduction (FIG. 8A). GL β has more flexibility to concentration but highest blocking efficiency was achieved at 5 μM concentration as well (FIG. 8B).

### Example 3

### Blood Total-RNA Sequencing

The Single-Cell Tagged Reverse Transcription (STRT) method with minor modifications was used to measure transcription initiation at the 5′-end of polyA+ transcripts starting from total blood-RNA as template [2]. Total blood-RNA samples were diluted to a concentration of 20 ng/μl and 2 μl were added to 4 μl GL mixture A that contained 1 M betaine, 2 mM dNTP mixture, 10 mM Tris-HCl (pH 8.0), 150 mM KCl, 0.2% Triton X-100, 2 μM equimolar barcoded 48-plex template switching oligonucleotides (Table 3, STRTv4-TSO1 to STRTv4-TSO48), and 5 μM GL α1/2 and β oligonucleotides. The blood-RNA samples (n=7, 20 ng/μl) were randomly placed in the 48-plex reaction plate and each one was sequenced with an individual barcode. The experiment was carried out in a 48-plex reaction plate. Total blood-RNA was denatured 30 s at 95° C. and incubated 10 min at 60° C. for GL hybridization. After incubation, RNA samples were incubated at 42° C. 5 μl GL mixture B was added to initiate cDNA synthesis. The 5 μl GL B mixture contained 1 M betaine, 100 mM Tris-HCl (pH 8.0), 10 mM DTT, 15 mM MgCl₂, 7 U RiboLOCK, 800 nM RNA-seq anchored poly-T primer with universal linker and 70 U RevertAid Premium reverse transcriptase. One microliter of ERCC mix 1 (Ambion) 1:1000 spike-in solution was used per whole 48-pled library. The samples were incubated 60 min at 42° C. and the reverse transcription enzyme was inactivated 5 min at 85° C.

The cDNAs were collected into one tube using 10% PEG-6000 and 0.9 M NaCl for purification and concentration purposes. The purified cDNA pool was first amplified using 14 cycles of PCR and 10 additional cycles to introduce the complete sets of adapters for Illumina sequencing. The libraries were size-selected (200-400 nt) using sequential AMPure XP bead selection protocol [3] and 0.7× and 0.22× ratios.

The sequences of the STRT libraries were pre-processed to (i) demultiplex the 48-plex samples based on internal 6 bp barcodes, (ii) exclude redundant reads to reduce PCR bias by unique molecular identifier (UMI) [4], (iii) align the reads to the human reference genome hg19 and spike-in sequences by TopHat software [5], (iv) quantify the expression levels in 50 nt strand-specific windows sliding in 25 nt steps, and (v) perform the basal quality check of the library and the sequencing. Then, we extracted the 50 nt windows, the expression levels of which were significantly fluctuated in the target samples larger than technical variations of spike-in RNAs. The data normalization was performed as previously described [6].

As a result of total blood RNA-seq, high-quality reads from the samples and from artificial spike RNA molecules were analyzed. RNA spike molecules were used for normalization purposes and as an internal control to track RNA degradation level during library preparation. The resulting spike-in 5′-end capture rate value of 89-91% indicated that 90% of all spike in molecules were intact and their 5′ ends were detected, see Table 1.

RNA sample endogenous 5′-end capture rate reflects the integrity of all mRNA sequences after GL treatment. The value 80-85% obtained according to the embodiments reflected that the mRNAs are in good condition after GL treatment and there was no significant degradation (n=7). In contrast, GLOBINclear™ resulted in a value of 53.8% indicating that the RNA was significantly degraded after globin reduction and only 53% of all mRNA molecules were still intact.

Globin molecules detection levels are listed in Table 2. After GL treatment and RNA-seq, globin α (HBA1/2) was the most prevalent transcript with its 4.71% (3′ DNA long and 3′ LNA, underlined in Table 2), Globin β had very low detection level having 0.48% (underlined in 3′ LNA in Table 2) prevalence. Based on RNA-seq data, naturally dominant globin α1/2 and β mRNAs were very efficiently blocked by GL prior cDNA synthesis and the globin molecules were no longer not highly dominating. Remember that untreated blood RNA consists of up to 64% of globin α1/2 and β, whereas GL treated blood RNA had a prevalence of globin α1/2 and β is 5.2%.

TABLE 1

| | Spike-in 5′-end | Endogenous 5′-end |
|---|---|---|
| | capture rate (%) | capture rate (%) |
| GLOBINclear ™ | 90.5 | 53.8 |
| GlobinLock | 89-91 | 80-85 |

TABLE 2

| Gene | Prevalence (%) | GL type |
|---|---|---|
| HBB | 42.17 | GL negative (control) |
| HBA1/2 | 21.42 | |
| RPLP2 | 1.16 | |
| S100A8 | 1.09 | |
| RPS27 | 0.94 | |
| HBB | 9.08 | 3′-5′ ends DNA |
| HBA1/2 | 8.88 | |
| RPLP2 | 2.64 | |
| S100A8 | 2.02 | |
| RPS27 | 1.71 | |
| HBA1/2 | 4.71 | 3′ end DNA long |
| S100A8 | 3.77 | |
| RPLP2 | 3.35 | |
| HBB | 2.55 | |
| RPL41 | 2.08 | |
| HBA1/2 | 7.17 | 3′ end DNA short |
| HBB | 4.40 | |
| RPLP2 | 3.85 | |
| S100A8 | 3.72 | |
| RPS27 | 1.98 | |
| S100A8 | 5.12 | 3′ LNA |
| HBA1/2 | 4.71 | |
| RPLP2 | 3.09 | |
| RPL41 | 2.16 | |
| RPS27 | 2.03 | |
| HBB | 0.48 | |
| HBB | 12.94 | 3′ ZNA |
| HBA1/2 | 9.85 | |
| RPLP2 | 2.66 | |
| RPS27 | 2.45 | |
| S100A8 | 2.43 | |

HBA1: gene encoding human hemoglobin α1
HBA2: gene encoding human hemoglobin α2
HBB: gene encoding human hemoglobin β
RPLP2: gene encoding human 60S acidic ribosomal protein P2
S100A8: gene encoding human S100 calcium binding protein A8
RPS27: gene encoding human 40S ribosomal protein S27
RPL41: gene encoding human 60S ribosomal protein L41

TABLE 3

| | Oligonucleotide | |
|---|---|---|
| | sequence at 5′-3′ | SEQ |
| Name | orientation | ID NO: |
| STRTv4-TSO1 | AAGCAGTGGTATCAACGCA | 34 |
| | GAGUNNNNCATGAT(GGG) | |
| STRTv4-TSO2 | AAGCAGTGGTATCAACGCA | 35 |
| | GAGUNNNNTATTGT(GGG) | |
| STRTv4-TSO3 | AAGCAGTGGTATCAACGCA | 36 |
| | GAGUNNNNCGTGGC(GGG) | |
| STRTv4-TSO4 | AAGCAGTGGTATCAACGCA | 37 |
| | GAGUNNNNAAAGTT(GGG) | |
| STRTv4-TSO5 | AAGCAGTGGTATCAACGCA | 38 |
| | GAGUNNNNTCTACC(GGG) | |
| STRTv4-TSO6 | AAGCAGTGGTATCAACGCA | 39 |
| | GAGUNNNNTACTTC(GGG) | |
| STRTv4-TSO7 | AAGCAGTGGTATCAACGCA | 40 |
| | GAGUNNNNCAAAGT(GGG) | |
| STRTv4-TSO8 | AAGCAGTGGTATCAACGCA | 41 |
| | GAGUNNNNATGCTT(GGG) | |
| STRTv4-TSO9 | AAGCAGTGGTATCAACGCA | 42 |
| | GAGUNNNNGTGGTA(GGG) | |
| STRTv4-TSO10 | AAGCAGTGGTATCAACGCA | 43 |
| | GAGUNNNNGCAGGA(GGG) | |
| STRTv4-TSO11 | AAGCAGTGGTATCAACGCA | 44 |
| | GAGUNNNNGGACAT(GGG) | |
| STRTv4-TSO12 | AAGCAGTGGTATCAACGCA | 45 |
| | GAGUNNNNAGGACT(GGG) | |
| STRTv4-TSO13 | AAGCAGTGGTATCAACGCA | 46 |
| | GAGUNNNNAGTTTA(GGG) | |
| STRTv4-TSO14 | AAGCAGTGGTATCAACGCA | 47 |
| | GAGUNNNNTGAAGC(GGG) | |
| STRTv4-TSO15 | AAGCAGTGGTATCAACGCA | 48 |
| | GAGUNNNNTAGCAT(GGG) | |
| STRTv4-TSO16 | AAGCAGTGGTATCAACGCA | 49 |
| | GAGUNNNNGTTGCC(GGG) | |
| STRTv4-TSO17 | AAGCAGTGGTATCAACGCA | 50 |
| | GAGUNNNNACGTTG(GGG) | |
| STRTv4-TSO18 | AAGCAGTGGTATCAACGCA | 51 |
| | GAGUNNNNTAAGGG(GGG) | |
| STRTv4-TSO19 | AAGCAGTGGTATCAACGCA | 52 |
| | GAGUNNNNGCCTAG(GGG) | |
| STRTv4-TSO20 | AAGCAGTGGTATCAACGCA | 53 |
| | GAGUNNNNCTCGCA(GGG) | |
| STRTv4-TSO21 | AAGCAGTGGTATCAACGCA | 54 |
| | GAGUNNNNGCGAAT(GGG) | |
| STRTv4-TSO22 | AAGCAGTGGTATCAACGCA | 55 |
| | GAGUNNNNGGGTTT(GGG) | |
| STRTv4-TSO23 | AAGCAGTGGTATCAACGCA | 56 |
| | GAGUNNNNGTAATG(GGG) | |
| STRTv4-TSO24 | AAGCAGTGGTATCAACGCA | 57 |
| | GAGUNNNNTAGAGA(GGG) | |
| STRTv4-TSO25 | AAGCAGTGGTATCAACGCA | 58 |
| | GAGUNNNNAGATGG(GGG) | |
| STRTv4-TSO26 | AAGCAGTGGTATCAACGCA | 59 |
| | GAGUNNNNATCTCT(GGG) | |
| STRTv4-TSO27 | AAGCAGTGGTATCAACGCA | 60 |
| | GAGUNNNNCGTATT(GGG) | |
| STRTv4-TSO28 | AAGCAGTGGTATCAACGCA | 61 |
| | GAGUNNNNACTTAT(GGG) | |
| STRTv4-TSO29 | AAGCAGTGGTATCAACGCA | 62 |
| | GAGUNNNNGATCTT(GGG) | |
| STRTv4-TSO30 | AAGCAGTGGTATCAACGCA | 63 |
| | GAGUNNNNGCTGTG(GGG) | |
| STRTv4-TSO31 | AAGCAGTGGTATCAACGCA | 64 |
| | GAGUNNNNACAATA(GGG) | |
| STRTv4-TSO32 | AAGCAGTGGTATCAACGCA | 65 |
| | GAGUNNNNTTCATA(GGG) | |
| STRTv4-TSO33 | AAGCAGTGGTATCAACGCA | 66 |
| | GAGUNNNNTTAACT(GGG) | |
| STRTv4-TSO34 | AAGCAGTGGTATCAACGCA | 67 |
| | GAGUNNNNATACAG(GGG) | |
| STRTv4-TSO35 | AAGCAGTGGTATCAACGCA | 68 |
| | GAGUNNNNAATAAA(GGG) | |
| STRTv4-TSO36 | AAGCAGTGGTATCAACGCA | 69 |
| | GAGUNNNNCTGTGT(GGG) | |
| STRTv4-TSO37 | AAGCAGTGGTATCAACGCA | 70 |
| | GAGUNNNNCCTAGA(GGG) | |
| STRTv4-TSO38 | AAGCAGTGGTATCAACGCA | 71 |
| | GAGUNNNNCCATCT(GGG) | |
| STRTv4-TSO39 | AAGCAGTGGTATCAACGCA | 72 |
| | GAGUNNNNGACACT(GGG) | |
| STRTv4-TSO40 | AAGCAGTGGTATCAACGCA | 73 |
| | GAGUNNNNTGGATG(GGG) | |
| STRTv4-TSO41 | AAGCAGTGGTATCAACGCA | 74 |
| | GAGUNNNNCTCCAT(GGG) | |
| STRTv4-TSO42 | AAGCAGTGGTATCAACGCA | 75 |
| | GAGUNNNNGAGATC(GGG) | |
| STRTv4-TSO43 | AAGCAGTGGTATCAACGCA | 76 |
| | GAGUNNNNTATGTA(GGG) | |
| STRTv4-TSO44 | AAGCAGTGGTATCAACGCA | 77 |
| | GAGUNNNNAACGCC(GGG) | |
| STRTv4-TSO45 | AAGCAGTGGTATCAACGCA | 78 |
| | GAGUNNNNCTTCTG(GGG) | |
| STRTv4-TSO46 | AAGCAGTGGTATCAACGCA | 79 |
| | GAGUNNNNGTCGGT(GGG) | |
| STRTv4-TSO47 | AAGCAGTGGTATCAACGCA | 80 |
| | GAGUNNNNATTTGC(GGG) | |
| STRTv4-TSO48 | AAGCAGTGGTATCAACGCA | 81 |
| | GAGUNNNNGAATTA(GGG) | |

NNNN is a random four nucleotides as unique molecular identifier
(GGG) represents three ribose guanine nucleotides

### Example 4

GlobinLock Efficiency by Quantitative PCR (qPCR)

During the cDNA synthesis, various GL oligonucleotides (SEQ ID NO: 8, 10; 84, 87; 82, 85; 4, 85; 83, 86) were added to the reaction mix in order to hamper RT of α and β gmRNA molecules. Another reaction mix contained nuclease-free water instead of GL and served as a negative control and a comparison for determining oligonucleotide masking effect with qPCR. Globin mRNA masking reactions were then carried out using GL-K+ buffer containing 1 μl 50% PEG-6000, 1.5 μl 3.2 M betaine, 0.4 μl 25 mM dNTP mixture, 0.375 μl 2 M KCl, 0.05 μl 1 M Tris-HCl (pH 8.0) and 0.05 μl 10% Triton X-100 per one blocking reaction. For α and β globin blocking, 0.12 μl of each specific GL oligonucleotide (100 μM) was added for one reaction, and equal amount of nuclease-free water was used for negative controls. Nuclease-free water was added up to 4 μl volume.

Two microlitres of whole-blood RNA (20 ng/μl) were added to previously prepared 4 μl GL-K+ buffer and hold on ice until denaturation. The gmRNA masking and cDNA synthesis was performed in 0.2 ml tubes using common thermocycler. Reaction conditions were following: 95° C. for 30 s as an initial denaturation, 60° C. for 10 min GL hybridization and 42° C. hold for loading of 5 μl RT mixture. The 5 μl RT mixture contained 2 μl nuclease-free water, 0.04 μl 100 μM T30VN (SEQ ID NO: 102), 1.6 μl 3.2 M betaine, 0.5 μl 1 M Tris-HCl (pH 8.0), 0.075 μl 1 M MgCl₂, 0.5 μl 100 mM DTT, 0.18 μl RiboLock RNase Inhibitor, and 0.13 μl RevertAid Premium Transcriptase. The concentrations were calculated for final RT volume in 10 μl, counting previous ingredients from GL-K+ buffer. Cycling parameters continued at 42° C. for 60 min RT reaction and 85° C. for 5 min for RT inactivation. Quantitative PCR was conducted using HOT FIREPol EvaGreen qPCR Mix Plus (ROX) with 7500 Fast Real-Time PCR instrument (Applied Biosystems) according to instructions where 200 nM primers (SEQ ID NO: 23-26) and 1 μl of template (cDNA) was used in 20 μl reaction volume. The primers were designed by Primer3 v4.0.0 software [10]. Specific product formation was ensured by performing a melt curve analysis to samples during real-time PCR program. Also, we run a conventional PCR for all globin primers and applied products on 2% TAE buffered agarose gel electrophoresis. All samples were additionally run with 10× diluted cDNA to enable quantification. Thermal program conditions were as follows: 95° C. for 15 min activation and then 30 cycles of 95° C. 15 s, 62° C. 20 s, 72° C. 30 s (at the end of which the fluorescence was measured). The qPCR data was analyzed using 7500 Software v2.0.5 (Applied Biosystems).

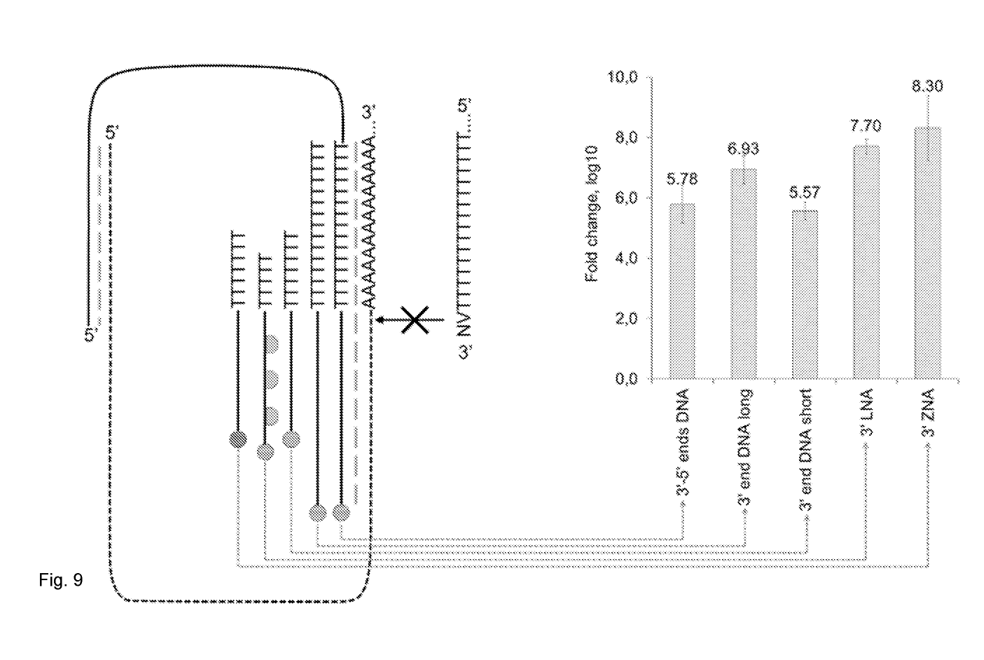

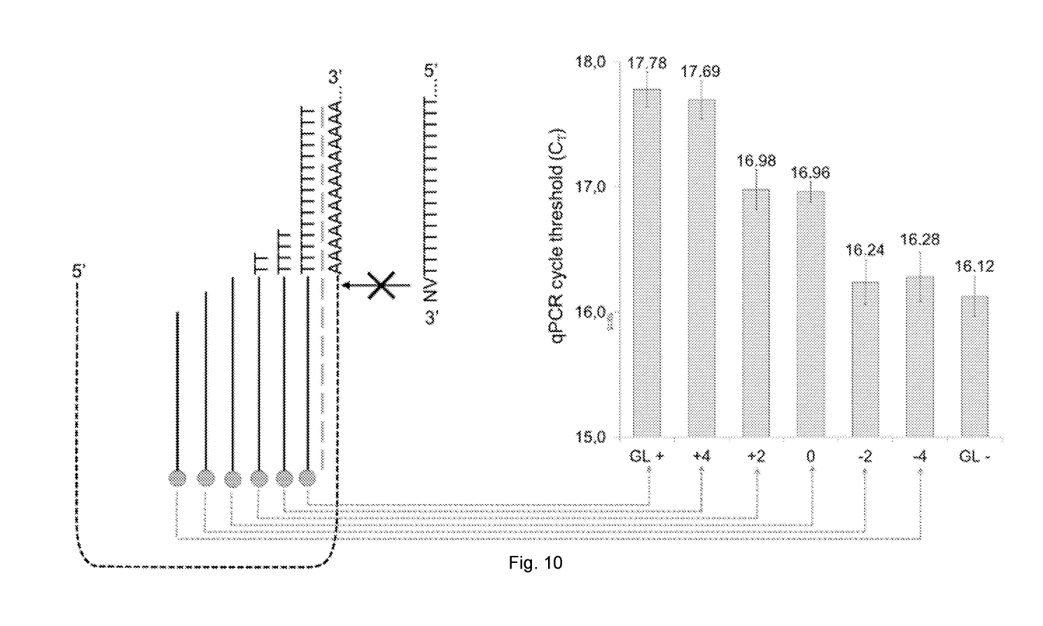

FIG. 9 illustrate the globin mRNA locking effect of five different blocking oligonucleotides; 3′-5′ ends DNA alpha (SEQ ID NO: 8); 3′-5′ ends DNA beta (SEQ ID NO: 10); 3′ end DNA long alpha (SEQ ID NO: 84); 3′ end DNA long beta (SEQ ID NO: 87); 3′ end DNA short alpha (SEQ ID NO: 82); 3′ end DNA short beta (SEQ ID NO: 85); 3′ ZNA alpha (SEQ ID NO: 4); 3′ ZNA beta (SEQ ID NO: 85); 3′ LNA alpha (SEQ ID NO: 83) and 3′ LNA beta (SEQ ID NO: 86). The fold change of globin cDNA synthesis was quantized by qPCR. The maximum efficiency was achieved by 3′ ZNA globin blocking oligonucleotides. The lowest effect was measured by 3′-end DNA short oligonucleotides.

### Example 5

### The Importance of Poly-T Stretch of Globin Blocking Oligonucleotides

This experiment was conducted as described in Example 4 but with difference of using human globin alpha blocking oligonucleotides of different lengths of the poly-A complementary sequence (SEQ ID NO: 169-173).

Different length of poly-T stretches were used to demonstrate the critical blocking region at gmRNA UTR and poly-A region. Most right-hand oligonucleotide GL+(SEQ ID NO: 84) has 15 nt poly-T region (T-15), which provides the maximum cycle threshold value (17.8) and highest blocking efficiency. The similar efficiency is achieved by 4-T nucleotides (SEQ ID NO: 169) but decreases with T-2 (SEQ ID NO: 170) and without any T nucleotide (SEQ ID NO: 171). If the 5′ end of the blocking oligonucleotide (SEQ ID NO: 172, 173) is located two or four nucleotides downstream of the poly-A tail, there is lack of blocking effect, providing similar cycle threshold value as the negative control (GL−).

### Example 6

### GlobinLock Effect by RNA-Seq

The modified STRT method was used. Artificial spike-in control mixture (ERCC Mix 1 (Ambion)) was diluted to 1:20 with nuclease free water and 2 μl were added to previously prepared 4 μl GL-K+ buffer. The buffer contained 1 M betaine, 2 mM dNTP mixture, 10 mM Tris (pH 8.0), 150 mM KCl, 0.2% Triton X-100, 2 μM equimolar barcoded 48-plex template switching oligonucleotides (SEQ ID NO: 103-150), and 5 μM different GL α and β oligonucleotides (SEQ ID NO: 8, 10; 84, 87; 82, 85; 4, 85; 83, 86). The RNA samples (n=7) were allocated in the 48-plex reaction plate and each one was sequenced with an individual barcode. After mixing GL-K+ and RNA on ice, the RNA was denatured 30 s at 95° C. and incubated 10 min at 60° C. for GL masking and continued 60 min at 42° C. Just after 60° C. incubation, the block was cooled to 42° C. where 5 μl of RT mixture was added to initiate cDNA synthesis. The RT mixture contained 1 M betaine, 50 mM Tris (pH 8.0), 5 mM DTT, 7.5 mM MgCl₂, RiboLOCK (0.7 U/μl), 400 nM T30VN (SEQ ID NO: 102) and RevertAid Premium reverse transcriptase (7 U/μl). The concentrations were calculated for final RT in 10 μl, counting previous ingredients from GL-K+ buffer. After 60 min RT reaction at 42° C. and enzyme 5 min inactivation at 85° C.

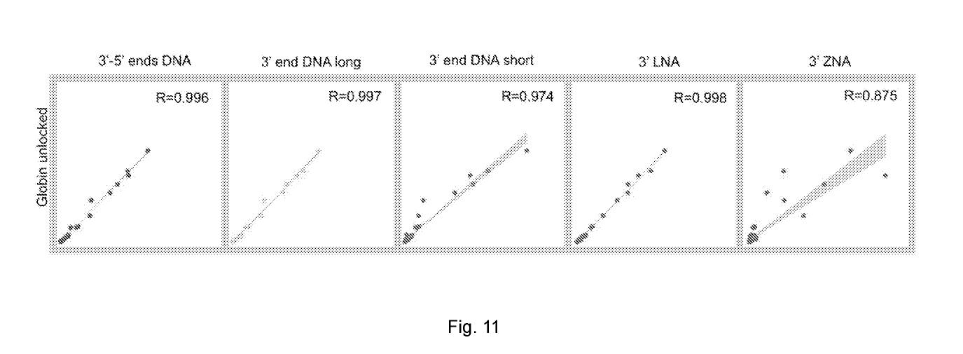

FIG. 11 illustrates specificity of different globin blocking oligonucleotides. Over all studied blocking conditions and oligonucleotides, ERCC spike-in 92 artificial mRNA-like molecules were analyzed simultaneously by RNA-sequencing method. The comparison was made against direct sequencing sample without any prior locking. Scatter plots over all detected artificial mRNAs demonstrated that the very high correlation between treated and untreated sample was achieved by 3′-5 ends DNA (SEQ ID NO: 8, 10), 3′ end DNA long (SEQ ID NO: 84, 87) and 3′ LNA (SEQ ID NO: 83, 86) oligonucleotides. The 3′ end DNA short (SEQ ID NO: 82, 85) has somehow lower correlation and 3′ ZNA (SEQ ID NO: 4, 85) has low correlation, which indicates low specificity to globin mRNAs. The grey area is confidence interval at 95%.

### Example 7

GlobinLock Efficiency by Quantitative PCR (qPCR)

During the cDNA synthesis, various concentration whole-blood RNA samples were added to the reaction. Another reaction mix contained nuclease-free water instead of GL and served as a negative control and a comparison for determining oligonucleotide masking effect with qPCR. Globin mRNA masking reactions were then carried out using GL-K+ buffer containing 1 μl 50% PEG-6000 (Sigma Aldrich), 1.5 μl 3.2 M betaine (Sigma), 0.4 μl 25 mM dNTP mixture (Thermo), 0.375 μl 2 M KCl (Sigma), 0.05 μl 1 M Tris-HCl (Sigma, pH 8.0) and 0.05 μl 10% Triton X-100 (Sigma) per one blocking reaction. For α and β globin blocking, 0.12 μl of each specific GL oligonucleotide (100 μM) (SEQ ID NO: 84, 87) was added for one reaction, and equal amount of nuclease-free water was used for negative controls. Nuclease-free water was added up to 4 μl volume.

Two microlitres of whole-blood RNA (0.5; 25; 50 ng/μl) were added to previously prepared 4 μl GL-K+ buffer and hold on ice until denaturation. The gmRNA masking and cDNA synthesis was performed in 0.2 ml tubes using common thermocycler. Reaction conditions were following: 95° C. for 30 s as an initial denaturation, 60° C. for 10 min GL hybridization and 42° C. hold for loading of 5 μl RT mixture. The 5 μl RT mixture contained 2 μl nuclease-free water, 0.04 μl 100 μM T30VN (SEQ ID NO: 102), 1.6 μl 3.2 M betaine, 0.5 μl 1 M Tris-HCl (pH 8.0), 0.075 μl 1 M MgCl₂ (Sigma), 0.5 μl 100 mM DTT (Sigma), 0.18 μl RiboLock RNase Inhibitor (Thermo), and 0.13 μl RevertAid Premium Transcriptase (Thermo). The concentrations were calculated for final RT volume in 10 μl, counting previous ingredients from GL-K+ buffer. Cycling parameters continued at 42° C. for 60 min RT reaction and 85° C. for 5 min for RT inactivation. Quantitative PCR was conducted using HOT FIREPol EvaGreen qPCR Mix Plus (ROX) (Solis Biodyne) with 7500 Fast Real-Time PCR instrument (Applied Biosystems) according to instructions where 200 nM primers (SEQ ID NO: 23-26) and 1 μl of template (cDNA) was used in 20 μl reaction volume. The primers were designed by Primer3 v4.0.0 software [10]. Specific product formation was ensured by performing a melt curve analysis to samples during real-time PCR program. Also, we run a conventional PCR for all globin primers and applied products on 2% TAE buffered agarose gel electrophoresis. All samples were additionally run with 10× diluted cDNA to enable quantification. Thermal program conditions were as follows: 95° C. for 15 min activation and then 30 cycles of 95° C. 15 s, 62° C. 20 s, 72° C. 30 s (at the end of which the fluorescence was measured). The qPCR data was analyzed using 7500 Software v2.0.5 (Applied Biosystems).

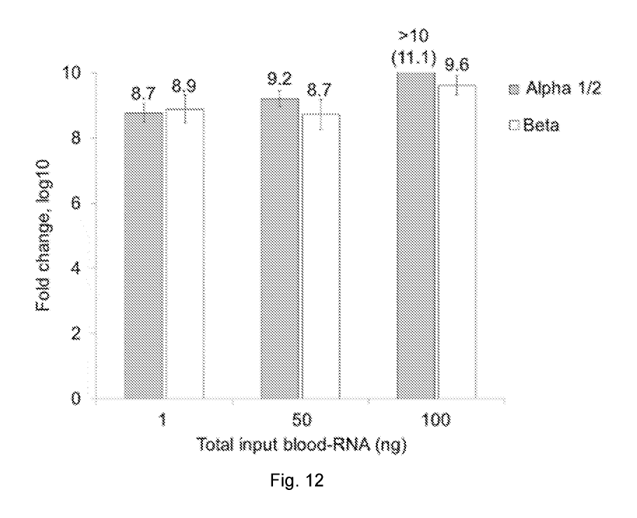

FIG. 12 illustrates globin mRNA blocking effect at different target concentrations. The 3′ end DNA long (SEQ ID NO: 84, 87) blocking oligonucleotides was used to measure the globin cDNA synthesis fold reduction at three different blood total-RNA input amounts; 1; 50 and 100 ng. Four RNA samples were analyzed simultaneously by qPCR. The blocking efficiency remains 8.7->10× and 8.7-9.6× in case of HBA and HBB, respectively.

### Example 8

GlobinLock Efficiency by Quantitative PCR (qPCR)

During the cDNA synthesis, various concentrations of 3′ end DNA long GL (SEQ ID NO: 84, 87) were added to the reaction mix in order to hamper RT of α and β gmRNA molecules. Another reaction mix contained nuclease-free water instead of GL and served as a negative control and a comparison for determining oligonucleotide masking effect with qPCR. Globin mRNA masking reactions were then carried out using GL-K+ buffer containing 1 μl 50% PEG-6000, 1.5 μl 3.2 M betaine, 0.4 μl 25 mM dNTP mixture, 0.375 μl 2 M KCl, 0.05 μl 1 M Tris-HCl (pH 8.0) and 0.05 μl 10% Triton X-100 per one blocking reaction. For α and β globin blocking, 0.12 μl of each specific GL oligonucleotide (100 μM) was added for one reaction, and equal amount of nuclease-free water was used for negative controls. Nuclease-free water was added up to 4 μl volume.

Two microlitres of whole-blood RNA (20 ng/μl) were added to previously prepared 4 μl GL-K+ buffer and hold on ice until denaturation. The gmRNA masking and cDNA synthesis was performed in 0.2 ml tubes using common thermocycler. Reaction conditions were following: 95° C. for 30 s as an initial denaturation, 60° C. for 10 min GL hybridization and 42° C. hold for loading of 5 μl RT mixture. The 5 μl RT mixture contained 2 μl nuclease-free water, 0.04 μl 100 μM T30VN (SEQ ID NO: 102), 1.6 μl 3.2 M betaine, 0.5 μl 1 M Tris-HCl (pH 8.0), 0.075 μl 1 M MgCl₂ (Sigma), 0.5 μl 100 mM DTT (Sigma), 0.18 μl RiboLock RNase Inhibitor (Thermo), and 0.13 μl RevertAid Premium Transcriptase (Thermo). The concentrations were calculated for final RT volume in 10 μl, counting previous ingredients from GL-K+ buffer. Cycling parameters continued at 42° C. for 60 min RT reaction and 85° C. for 5 min for RT inactivation. Quantitative PCR was conducted using HOT FIREPol EvaGreen qPCR Mix Plus (ROX) (Solis Biodyne) with 7500 Fast Real-Time PCR instrument (Applied Biosystems) according to instructions where 200 nM primers (SEQ ID NO: 23-26) and 1 μl of template (cDNA) was used in 20 μl reaction volume. The primers were designed by Primer3 v4.0.0 software [10]. Specific product formation was ensured by performing a melt curve analysis to samples during real-time PCR program. Also, we run a conventional PCR for all globin primers and applied products on 2% TAE buffered agarose gel electrophoresis. All samples were additionally run with 10× diluted cDNA to enable quantification. Thermal program conditions were as follows: 95° C. for 15 min activation and then 30 cycles of 95° C. 15 s, 62° C. 20 s, 72° C. 30 s (at the end of which the fluorescence was measured). The qPCR data was analyzed using 7500 Software v2.0.5 (Applied Biosystems).

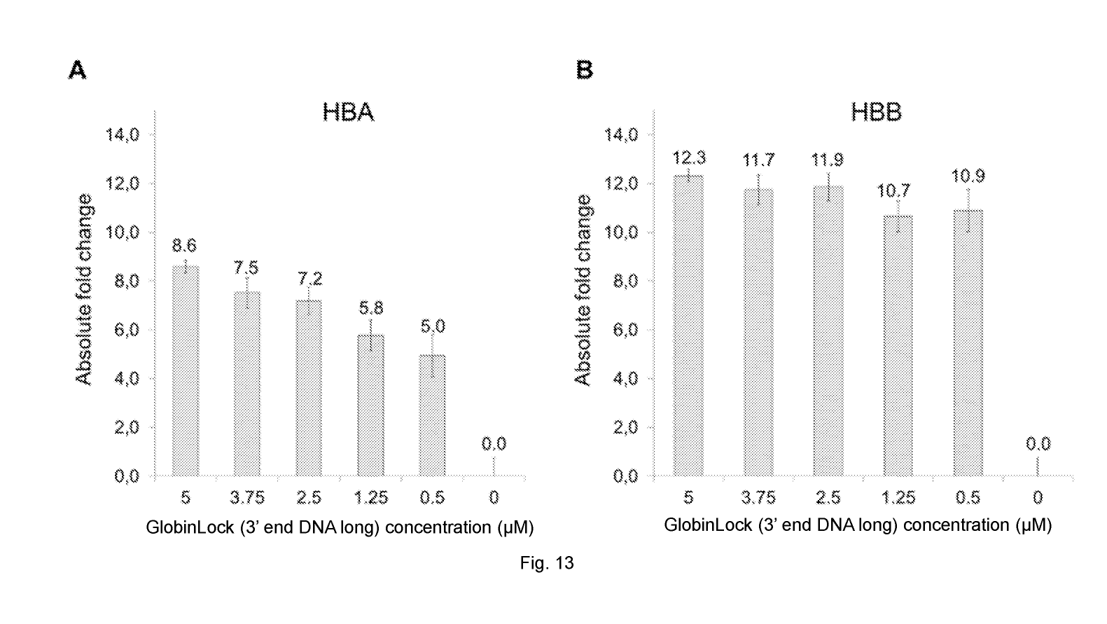

FIGS. 13A and 13B illustrate globin locking effect at different blocking oligonucleotide concentration 0.5-5 μM. The 3′ end DNA long blocking oligonucleotides (SEQ ID NO: 84, 87) were used to observe the locking efficiency at different oligonucleotide concentration by qPCR. Human HBA locking was concentration dependent, varying significantly at 0.5 μM and 5 μM concentration. The locking of HBB was more robust, enabling efficient locking even at relatively low oligonucleotide concentration (0.5 μM).

### Example 9

### GlobinLock Effect by RNA-Sequencing

The modified STRT method was used. Human whole-blood RNA samples were diluted with RNase-DNase-free water to concentration 30 ng/μl, and 2 μl was added to previously prepared 4 μl GL-K+ buffer. The buffer contained 1 M betaine, 2 mM dNTP mixture, 10 mM Tris (pH 8.0), 150 mM KCl, 0.2% Triton X-100, 2 μM equimolar barcoded 48-plex template switching oligonucleotides (SEQ ID NO: 103-150), and 5 μM different GL α and β oligonucleotides (SEQ ID NO: 8, 10; 84, 87; 82, 85; 4, 85; 83, 86). The RNA samples (n=7) were allocated in the 48-plex reaction plate and each one was sequenced with an individual barcode. After mixing GL-K+ and RNA on ice, the RNA was denatured 30 s at 95° C. and incubated 10 min at 60° C. for GL masking and continued 60 min at 42° C. Just after 60° C. incubation, the block was cooled to 42° C. where 5 μl of RT mixture was added to initiate cDNA synthesis. The RT mixture contained 1 M betaine, 50 mM Tris (pH 8.0), 5 mM DTT, 7.5 mM MgCl2, RiboLOCK (0.7 U/μl), 400 nM T30VN (SEQ ID NO: 102) and RevertAid Premium reverse transcriptase (7 U/μl). The concentrations are calculated for final RT in 10 μl, counting previous ingredients from GL-K+ buffer. Two microlitres of ERCC Mix 1 (Ambion) 1:500 spike-in dilution with nuclease-free water was used per whole 48-plex library. After 60 min RT reaction at 42° C. and enzyme 5 min inactivation at 85° C.

All 48 reaction volumes were pooled into 2.0 ml tube (˜500 μl). Dynabeads MyOne C1 Streptavidin (Invitrogen) were washed twice and used to capture the formed cDNA molecules (and free VN30 primers). For the capture, 100 μl of the beads were prepared and coupled according to the instructions. After three rounds of EB (10 mM Tris, pH 8.0) and one round of water washing, the DNA enriched beads were suspended in 75 μl water and incubated at 75° C. three minutes to release biotin from streptavidin beads. The supernatant was used as a template for further full cDNA amplification as described in [9]. The purified cDNA pool was first amplified using 14 cycles of PCR and 15 additional cycles to introduce the complete sets of adapters for Illumina sequencing. The libraries were size-selected (200-400 bp) using sequential AMPure XP bead selection protocol as described in [9].

### RNA-Sequencing Data Analysis

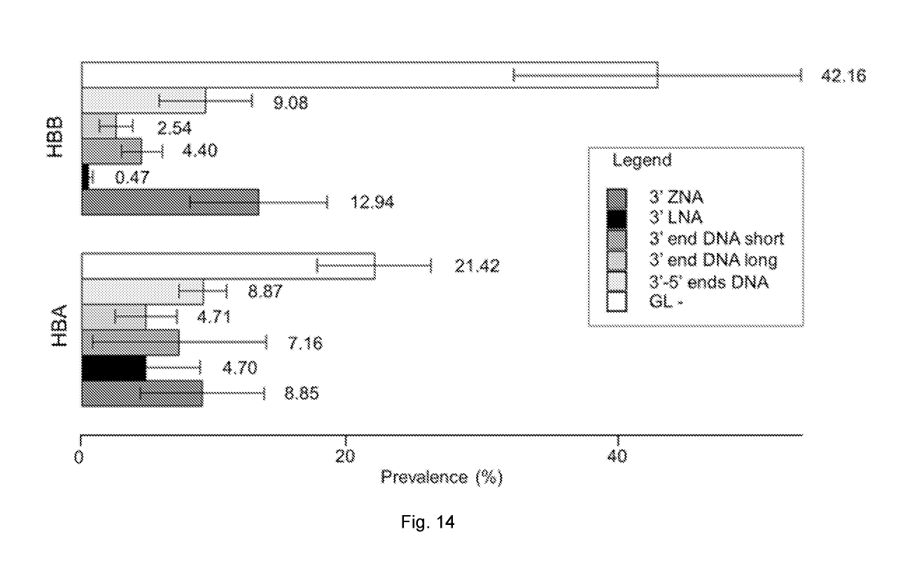

Preprocessing of the RNA-seq raw sequences, alignment and quantitation were performed by STRTprep pipeline ([9]; https://github.com/shka/STRTprep). Although the pipeline uses only uniquely mapped reads, two loci HBA1 and HBA2 are highly similar. Therefore, the HBA2 locus and the upstream up to 500 nt (chr16:222346 . . . 223709 on hg19 reference genome) were masked before alignment. Branch v3dev (commit 698fa8c . . . ) was used as the standard procedure with PCR-bias reduction based on the unique molecular identifier (UMI), and branch v3devNoUMI (commit e0d6721 . . . ) was as a special procedure which skips the reduction step. FIG. 14 was generated using R version 3.2.2.

FIG. 14 illustrates globin reduction effect by different blocking oligonucleotides. The locking efficiency was measured by RNA-sequencing over seven different total blood-RNA samples. After library preparation and sequencing, proper software was used to count the reads per all detected genes, e.g. HBA and HBB. As HBA1 and HBA2 has one nucleotide difference at mRNA 50 nt 5′ end, and one mapping mismatch was allowed, the HBA2 was masked in the reference to enable proper mapping. The masking was required to avoid mapping on two targets simultaneously, which was not allowed by used software, causing total discarding of HBA1 and HBA2 reads.

White bars represent direct RNA sequencing without any locking. Grey scale to black correspond to different blocking oligonucleotides, providing the prevalence percent over all normalized read counts of analyzed seven total blood-RNA samples. Globin lock negative control provides 42% HBB reads over all mapped reads. The lower prevalence was detected by 3′ LNA blocking oligonucleotides (SEQ ID NO: 86), having 0.5% prevalence. The HBA prevalence of globin lock negative was 21% and the lowest value was detected by 3′ LNA (SEQ ID NO: 83) and 3′ end DNA long (SEQ ID NO: 84) blocking oligonucleotides, having 4.7% prevalence.

Based on previous, globin (HBA and HBB) total read count was 64% in case of direct RNA-sequencing of wbRNA samples. Highest gmRNA locking efficiency was achieved by combination of 3′ LNA and 3′ end DNA long blocking oligonucleotides, which bind with HBB and HBA, respectively. In that case, the total globin prevalence drops from 63.6% to 5.2%, providing 10× globin cDNA synthesis reduction.

### Example 10

GlobinLock Efficiency by Quantitative PCR (qPCR)

Whole-blood RNA from different species were tested by qPCR. Globin mRNA masking reactions were then carried out using GL-K+ buffer containing 1 μl 50% PEG-6000, 1.5 μl 3.2 M betaine, 0.4 μl 25 mM dNTP mixture, 0.375 μl 2 M KCl, 0.05 μl 1 M Tris-HCl (pH 8.0) and 0.05 μl 10% Triton X-100 per one blocking reaction. For α and β globin blocking, 0.12 μl of each specific GL oligonucleotide (100 μM) (SEQ ID NO: 88-101) was added for one reaction, and equal amount of nuclease-free water was used for negative controls. Nuclease-free water was added up to 4 μl volume.

Two microlitres of whole-blood RNA (20 ng/μl) from different species were added to previously prepared 4 μl GL-K+ buffer and hold on ice until denaturation. The gmRNA masking and cDNA synthesis was performed in 0.2 ml tubes using common thermocycler. Reaction conditions were following: 95° C. for 30 s as an initial denaturation, 60° C. for 10 min GL hybridization and 42° C. hold for loading of 5 μl RT mixture. The 5 μl RT mixture contained 2 μl nuclease-free water, 0.04 μl 100 μM T30VN (SEQ ID NO: 102), 1.6 μl 3.2 M betaine, 0.5 μl 1 M Tris-HCl (pH 8.0), 0.075 μl 1 M MgCl₂, 0.5 μl 100 mM DTT, 0.18 μl RiboLock RNase Inhibitor, and 0.13 μl RevertAid Premium Transcriptase. The concentrations were calculated for final RT volume in 10 μl, counting previous ingredients from GL-K+ buffer. Cycling parameters continued at 42° C. for 60 min RT reaction and 85° C. for 5 min for RT inactivation. Quantitative PCR was conducted using HOT FIREPol EvaGreen qPCR Mix Plus (ROX) (Solis Biodyne) with 7500 Fast Real-Time PCR instrument (Applied Biosystems) according to instructions where 200 nM primers (SEQ ID NO: 151-168) and 1 μl of template (cDNA) was used in 20 μl reaction volume. The primers were designed by Primer3 v4.0.0 software [10]. Specific product formation was ensured by performing a melt curve analysis to samples during real-time PCR program. Also, we run a conventional PCR for all globin primers and applied products on 2% TAE buffered agarose gel electrophoresis. All samples were additionally run with 10× diluted cDNA to enable quantification. Thermal program conditions were as follows: 95° C. for 15 min activation and then 30 cycles of 95° C. 15 s, 62° C. 20 s, 72° C. 30 s (at the end of which the fluorescence was measured). The qPCR data was analyzed using 7500 Software v2.0.5 (Applied Biosystems).

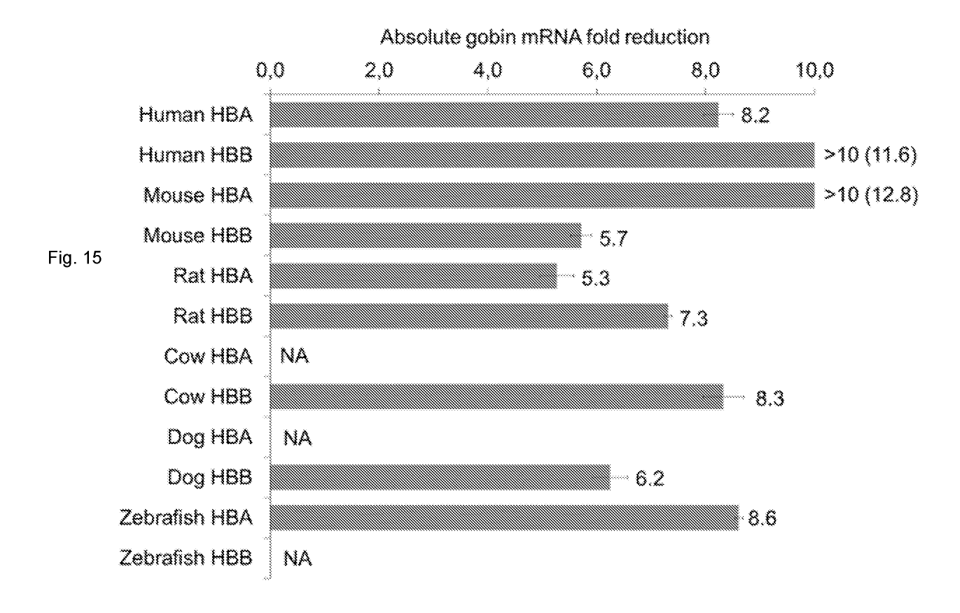

FIG. 15 illustrates the prevalence of globin mRNAs (HBA and HBB) from different species was reduced by type 3′ end DNA long blocking oligonucleotides and quantified by qPCR. NA means the inability to detect specific globin with unique primers using SYBR green qPCR assay.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

[1] Mastrokolias et al., Increased sensitivity of next generation sequencing-based expression profiling after globin reduction in human blood RNA, BMC Genomics 2012, 13:28
[2] Islam et al., Highly multiplexed and strand-specific single-cell RNA 5′ end sequencing, Nature Protocols 2012, 7(5): 813-828
[3] New England BioLabs® Inc., Size Selection (E6270), www.neb.com/protocols/1/01/01/size-selection-e6270, retrieved on May 7, 2015
[4] Kivioja et al., Counting absolute numbers of molecules using unique molecular identifiers, Nature Methods 2011, 9(1): 72-74
[5] Trapnell et al., TopHat: discovering splice junctions with RNA-Seq, Bioinformatics 2009, 25(9): 1105-1111
[6] Katayama et al., SAMstrt: statistical test for differential expression in single-cell transcriptome with spike-in normalization, Bioinformatics 2013, 29(22): 2943-2945
[7] Zucker, Mfold web server for nucleic acid folding and hybridization prediction, Nucleic Acids Research 2003, 31(13): 3406-3415
[8] Gruber et al., The Vienna RNA websuite, Nucleic Acids Research 2008, 36(Web server issue): W70-W74
[9] Krjuškov et al., Single-cell transcriptome analysis of endometrial tissue, Hum Reprod. 2016, 31(4): 844-853
[10] Koressaar and Remm, Enhancements and modifications of primer design program Primer3, Bioinformatics 2007, 23(10): 1289-1291

1.-21. (canceled)

22. A blocking oligonucleotide comprising, from a 3′-end towards a 5′-end of said blocking oligonucleotide:
a 3′-end complementary sequence complementary to a 3′-end sequence of a globin messenger ribonucleic acid (mRNA molecule); and
a poly-A complementary sequence of at least one nucleotide complementary to at least a portion of a poly-A sequence of said globin mRNA molecule, wherein said blocking oligonucleotide is capable of inhibiting binding of a reverse transcription anchored poly-T primer to said globin mRNA molecule.

23. The blocking oligonucleotide according to claim 22, wherein a 3′-end of said blocking oligonucleotide is chemically modified to inhibit enzymatic extension of said blocking oligonucleotide.

24. The blocking oligonucleotide according to claim 23, wherein said 3′-end of said blocking oligonucleotide is phosphorylated to inhibit enzymatic extension of said blocking oligonucleotide.

25. The blocking oligonucleotide according to claim 23, wherein said 3′-end of said blocking oligonucleotide comprises a Zip nucleotide to inhibit enzymatic extension of said blocking oligonucleotide.

26. The blocking oligonucleotide according to claim 22, wherein said 3′-end complementary sequence consists of 10 to 100 nucleotides complementary to said 3′-end sequence of said globin mRNA molecule.

27. The blocking oligonucleotide according to claim 26, wherein said 3′-end complementary sequence consists of 15 to 60 nucleotides complementary to said 3′-end sequence of said globin mRNA molecule.

28. The blocking oligonucleotide according to claim 27, wherein said 3′-end complementary sequence consists of 25 to 35 nucleotides complementary to said 3′-end sequence of said globin mRNA molecule.

29. The blocking oligonucleotide according to claim 22, wherein said poly-A complementary sequence comprises at least four nucleotides complementary to at least a portion of said poly-A sequence of said globin mRNA molecule.

30. The blocking oligonucleotide according to claim 22, wherein said poly-A complementary sequence consists of 1 to 50 nucleotides complementary to said at least a portion of said poly-A sequence of said globin mRNA molecule.

31. The blocking oligonucleotide according to claim 30, wherein said poly-A complementary sequence consists of 2 to 30 nucleotides complementary to said at least a portion of said poly-A sequence of said globin mRNA molecule.

32. The blocking oligonucleotide according to claim 31, wherein said poly-A complementary sequence consists of 4 to 20 nucleotides complementary to said at least a portion of said poly-A sequence of said globin mRNA molecule.

33. The blocking oligonucleotide according to claim 22, wherein said 3′-end complementary sequence comprises at least one locked nucleic acid (LNA) nucleotide.

34. The blocking oligonucleotide according to claim 33, further comprising, from said 3′-end towards said 5′-end of said blocking oligonucleotide:
a linker sequence; and
a 5′-end complementary sequence complementary to a 5′-end sequence of said globin mRNA molecule.

35. The blocking oligonucleotide according to claim 34, wherein said 5′-end complementary sequence consists of 10 to 40 nucleotides complementary to said 5′-end sequence of said globin mRNA molecule.

36. The blocking oligonucleotide according to claim 34, wherein said linker sequence consists of 30 to 80 nucleotides.

37. The blocking oligonucleotide according to claim 34, wherein said blocking oligonucleotide and said globin mRNA molecule form a circular complex when said 5′-end complementary sequence is hybridized to said 5′-end sequence of said globin mRNA molecule, said 3′-end complementary sequence is hybridized to said 3′-end sequence of said globin mRNA molecule and said poly-A complementary sequence is hybridized to said at least a portion of said poly-A sequence of said globin mRNA molecule.

38. The blocking oligonucleotide according to claim 22, wherein said globin mRNA molecule is a globin α mRNA molecule and said blocking oligonucleotide comprises the sequence of 5′-TTTTTTTGCYGCCCACTCAGACTTTA-3′ (SEQ ID NO: 82), wherein Y denotes T or C.

39. The blocking oligonucleotide according to claim 38, wherein said blocking oligonucleotide comprises a sequence selected from the group consisting of:
i) 5′-TTTTTTTGCYGCCCACTCAGACTTTA-3′ (SEQ ID NO: 82);
ii) 5′-TTTTTG+CYGCCC+ACTCAG+ACTTTA+TTC-3′ (SEQ ID NO: 83), wherein +C, +A and +T denote locked nucleic acid (LNA) nucleotides;
iii) 5′-TTTTTTTTTTTTGCYGCCCACTCAGACTTTATTCAAAGAC-3′ (SEQ ID NO: 4);
iv) 5′-TTTTTTTTTTTTTTTGCYGCCCACTCAGACTTTATTCAAAGACCA-3′ (SEQ ID NO: 84);
v) 5′-CGCGAGCGCGCCAGGGTTTATG(Xₙ)_{N}TTTTTTTTTTTTTTTGCYGCCC ACTCAGACTTTATTCAAAGAC-3′; wherein Xₙ is A, T, G, C or U, n=1-N and N is from 40 to 60;
vi) 5′-CGCGAGCGCGCCAGGGTTTATGTAATTAGAATTAGAATGAATAGC TAACCTGATATGTTGAAGAACTATGACAGACATTTTTTTTTTTTTTTGCYGCCCACTC AGACTTTATTCAAAGAC-3′ (SEQ ID NO: 8); and
vii) variants of said sequences i) to vi) in which a nucleotide at a 3′-end is phosphorylated or is a Zip nucleotide.

40. The blocking oligonucleotide according to claim 22, wherein said globin mRNA molecule is a globin β mRNA molecule and said blocking oligonucleotide comprises the sequence of 5′-TTTTTTTTTTTTGCAATGAAAATAAATGTTTTTTATTAGG-3′ (SEQ ID NO: 85).

41. The blocking oligonucleotide according to claim 34, wherein said blocking oligonucleotide comprises a sequence selected from the group consisting of:
i) 5′-TTTTTTTTTTTTGCAATGAAAATAAATGTTTTTTATTAGG-3′ (SEQ ID NO: 85);
ii) 5′-TTTTTTTTTTG+CAATGA+AAATAA+ATGTTT+TTTATTAGG-3′ (SEQ ID NO: 86), wherein +C, +A and +T denote locked nucleic acid (LNA) nucleotides;
iii) 5′-TTTTTTTTTTTTTTTGCAATGAAAATAAATGTTTTTTATTAGGCAG AATCCAGAT-3′ (SEQ ID NO: 87);
iv) 5′-TTTTTTTTTTTTTTTGCAATGAAAATAAATGTTTTTTATTAGGCAG-3′ (SEQ ID NO: 5);
v) 5′-AGTGAACACAGTTGTGTCAGAAGCAAATGT(Xₘ)_{M}TTTTTTTTTTTTT TTGCAATGAAAATAAATGTTTTTTATTAGGCAG-3′, wherein Xₘ is A, T, G or C, m=1-M and M is from 40 to 60;
vi) 5′-AGTGAACACAGTTGTGTCAGAAGCAAATGTAGAATGAATAGCTAA CCTGATATGTTGAAGAACTATGACAGACCTTTTTTTTTTTTTTTGCAATGAAAATAAA TGTTTTTTATTAGGCAG-3′ (SEQ ID NO: 10); and
vii) variants of said sequences i) to vi) in which a nucleotide at a 3′-end is phosphorylated or is a Zip nucleotide.

42. A kit for producing a complementary deoxyribonucleic acid, cDNA, molecule comprising:
at least one blocking oligonucleotide according to claim 22; and
at least one reverse transcription anchored poly-T primer.

43. The kit according to claim 42, further comprising a reverse transcription enzyme.

44. A double strand complex comprising:
a globin messenger ribonucleic acid (mRNA) molecule; and
a blocking oligonucleotide according to claim 22 hybridized to at least a portion of said globin mRNA molecule.

45. A sample comprising:
at least one RNA molecule; and
a double strand complex according to claim 44.

46. A method of producing a complementary deoxyribonucleic acid (cDNA) molecule comprising the steps of:
(a) contacting a sample comprising at least one messenger ribonucleic acid, mRNA, molecule and at least one globin messenger RNA (mRNA) molecule with at least one blocking oligonucleotide according to claim 22 under conditions enabling hybridization of a blocking oligonucleotide of said at least one blocking oligonucleotide to a globin mRNA molecule of said at least one globin mRNA molecule;
(b) adding a reverse transcription anchored poly-T primer to said sample; and
(c) adding a reverse transcription enzyme to said sample to produce said cDNA molecule from said at least one mRNA molecule.

47. The method according to claim 46, wherein step (a) comprises contacting said sample with
(i) at least one first blocking oligonucleotide comprising, from a 3′-end towards a 5′-end of said blocking oligonucleotide:
a 3′-end complementary sequence complementary to a 3′-end sequence of a globin messenger ribonucleic acid (mRNA molecule); and
a poly-A complementary sequence of at least one nucleotide complementary to at least a portion of a poly-A sequence of said globin mRNA molecule, wherein said first blocking oligonucleotide is capable of inhibiting binding of a reverse transcription anchored poly-T primer to said globin mRNA molecule, and wherein said globin mRNA molecule is a globin α mRNA molecule and said first blocking oligonucleotide comprises the sequence of 5′-TTTTTTTGCYGCCCACTCAGACTTTA-3′ (SEQ ID NO: 82), wherein Y denotes T or C, and
(ii) at least one second blocking oligonucleotide comprising, from a 3′-end towards a 5′-end of said blocking oligonucleotide:
a 3′-end complementary sequence complementary to a 3′-end sequence of a globin messenger ribonucleic acid (mRNA molecule); and
a poly-A complementary sequence of at least one nucleotide complementary to at least a portion of a poly-A sequence of said globin mRNA molecule, wherein said second blocking oligonucleotide is capable of inhibiting binding of a reverse transcription anchored poly-T primer to said globin mRNA molecule, and wherein said globin mRNA molecule is a globin β mRNA molecule and said second blocking oligonucleotide comprises the sequence of 5′-TTTTTTTTTTTTGCAATGAAAATAAATGTTTTTTATTAGG-3′ (SEQ ID NO: 85).

48. The method according to claim 46, further comprising:
(a1) heating said sample to denature RNA secondary structures following step (a) but prior to step (c); and
(a2) cooling said sample from step (a1) to a hybridization temperature for said at least one blocking oligonucleotide prior to step (c).


SEQUENCE LISTING (173 sequences)

SEQ ID NO: 1 - DNA, 31 bp, Artificial Sequence
  Primer for reverse transcription
  misc_feature (31)..(31): n is a, c, g, or t
tttttttttttttttttttttttttttttvn

SEQ ID NO: 2 - DNA, 28 bp, Artificial Sequence
  Complementary to 3′-end sequence of human globin alpha mRNA
gcygcccactcagactttattcaaagac

SEQ ID NO: 3 - DNA, 31 bp, Artificial Sequence
  Complementary to 3′-end sequence of human globin beta mRNA
gcaatgaaaataaatgttttttattaggcag

SEQ ID NO: 4 - DNA, 43 bp, Artificial Sequence
  Complementary to 3′-end sequence and poly-A tail of human globin alpha mRNA
tttttttttttttttgcygcccactcagactttattcaaagac

SEQ ID NO: 5 - DNA, 46 bp, Artificial Sequence
  Complementary to 3′-end sequence and poly-A tail of human globin beta mRNA
tttttttttttttttgcaatgaaaataaatgttttttattaggcag

SEQ ID NO: 6 - DNA, 22 bp, Artificial Sequence
  Complementary to 5′-end sequence of human globin alpha mRNA
cgcgagcgcgccagggtttatg

SEQ ID NO: 7 - DNA, 30 bp, Artificial Sequence
  Complementary to 5′-end sequence of human globin beta mRNA
agtgaacacagttgtgtcagaagcaaatgt

SEQ ID NO: 8 - DNA, 120 bp, Artificial Sequence
  Blocking oligonucleotide for human globin alpha mRNA
cgcgagcgcgccagggtttatgtaattagaattagaatgaatagctaacctgatatgttgaagaactatgacagacatttttttttttttttgcygcccactcagactttattcaaagac

SEQ ID NO: 9 - DNA, 120 bp, Artificial Sequence; identical to SEQ ID NO: 8
  Blocking oligonucleotide for human globin alpha mRNA
  modified_base (120)..(120): Phosphorylated nucleotide

SEQ ID NO: 10 - DNA, 120 bp, Artificial Sequence
  Blocking oligonucleotide for human globin beta mRNA
agtgaacacagttgtgtcagaagcaaatgtagaatgaatagctaacctgatatgttgaagaactatgacagacctttttttttttttttgcaatgaaaataaatgttttttattaggcag

SEQ ID NO: 11 - DNA, 120 bp, Artificial Sequence; identical to SEQ ID NO: 10
  Blocking oligonucleotide for human globin beta mRNA
  modified_base (120)..(120): Phosphorylated nucleotide

SEQ ID NO: 12 - RNA, 40 nt, Homo sapiens
uggucuuugaauaaagucugagugggcggcaaaaaaaaaa

SEQ ID NO: 13 - DNA, 40 bp, Artificial Sequence
  Complementary to 3′-end sequence and poly-A tail of human globin alpha1 mRNA
ttttttttttgccgcccactcagactttattcaaagacca

SEQ ID NO: 14 - RNA, 40 nt, Homo sapiens
uggucuuugaauaaagucugagugggcagcaaaaaaaaaa

SEQ ID NO: 15 - DNA, 40 bp, Artificial Sequence
  Complementary to 3′-end sequence and poly-A tail of human globin alpha2 mRNA
ttttttttttgctgcccactcagactttattcaaagacca

SEQ ID NO: 16 - DNA, 40 bp, Artificial Sequence
  Complementary to 3′-end sequence and poly-A tail of human globin alpha mRNA
ttttttttttgcygcccactcagactttattcaaagacca

SEQ ID NO: 17 - RNA, 41 nt, Homo sapiens
cugccuaauaaaaaacauuuauuuucauugcaaaaaaaaaa

SEQ ID NO: 18 - DNA, 41 bp, Artificial Sequence
  Complementary to 3′-end sequence and poly-A tail of human globin beta mRNA
ttttttttttgcaatgaaaataaatgttttttattaggcag

SEQ ID NO: 19 - RNA, 623 nt, Homo sapiens
cauaaacccuggcgcgcucgcggcccggcacucuucugguccccacagacucagagagaacccaccauggugcugucuccugccgacaagaccaacgucaaggccgccugggguaaggucggcgcgcacgcuggcgaguauggugcggaggcccuggagaggauguuccuguccuuccccaccaccaagaccuacuucccgcacuucgaccugagccacggcucugcccagguuaagggccacggcaagaagguggccgacgcgcugaccaacgccguggcgcacguggacgacaugcccaacgcgcuguccgcccugagcgaccugcacgcgcacaagcuucggguggacccggucaacuucaagcuccuaagccacugccugcuggugacccuggccgcccaccuccccgccgaguucaccccugcggugcacgccucccuggacaaguuccuggcuucugugagcaccgugcugaccuccaaauaccguuaagcuggagccucgguggccaugcuucuugccccuugggccuccccccagccccuccuccccuuccugcacccguacccccguggucuuugaauaaagucugagugggcggcaaaaaaaaaaaaaaaaaa

SEQ ID NO: 20 - RNA, 622 nt, Homo sapiens
cauaaacccuggcgcgcucgcgggccggcacucuucugguccccacagacucagagagaacccaccauggugcugucuccugccgacaagaccaacgucaaggccgccugggguaaggucggcgcgcacgcuggcgaguauggugcggaggcccuggagaggauguuccuguccuuccccaccaccaagaccuacuucccgcacuucgaccugagccacggcucugcccagguuaagggccacggcaagaagguggccgacgcgcugaccaacgccguggcgcacguggacgacaugcccaacgcgcuguccgcccugagcgaccugcacgcgcacaagcuucggguggacccggucaacuucaagcuccuaagccacugccugcuggugacccuggccgcccaccuccccgccgaguucaccccugcggugcacgccucccuggacaaguuccuggcuucugugagcaccgugcugaccuccaaauaccguuaagcuggagccucgguagccguuccuccugcccgcugggccucccaacgggcccuccuccccuccuugcaccggcccuuccuggucuuugaauaaagucugagugggcagcaaaaaaaaaaaaaaaaaa

SEQ ID NO: 21 - RNA, 644 nt, Homo sapiens
acauuugcuucugacacaacuguguucacuagcaaccucaaacagacaccauggugcaucugacuccugaggagaagucugccguuacugcccuguggggcaaggugaacguggaugaaguugguggugaggcccugggcaggcugcugguggucuacccuuggacccagagguucuuugaguccuuuggggaucuguccacuccugaugcuguuaugggcaacccuaaggugaaggcucauggcaagaaagugcucggugccuuuagugauggccuggcucaccuggacaaccucaagggcaccuuugccacacugagugagcugcacugugacaagcugcacguggauccugagaacuucaggcuccugggcaacgugcuggucugugugcuggcccaucacuuuggcaaagaauucaccccaccagugcaggcugccuaucagaaagugguggcugguguggcuaaugcccuggcccacaaguaucacuaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcaaaaaaaaaaaaaaaaaa

SEQ ID NO: 22 - DNA, 58 bp, Artificial Sequence
  Reverse transcription anchored poly-T primer
  modified_base (1)..(1): Amine at 5′-end
  misc_feature (58)..(58): n is a, c, g, or t
gtgatcttgaacagagtgtcagtgcgatttttttttttttttttttttttttttttvn

SEQ ID NO: 23 - DNA, 23 bp, Artificial Sequence
  qPCR globin alpha detection primer, left
actcttctggtccccacagactc

SEQ ID NO: 24 - DNA, 22 bp, Artificial Sequence
  qPCR globin alpha detection primer, right
agtaggtcttggtggtggggaa

SEQ ID NO: 25 - DNA, 22 bp, Artificial Sequence
  qPCR globin beta detection primer, left
ctttgccacactgagtgagctg

SEQ ID NO: 26 - DNA, 28 bp, Artificial Sequence
  qPCR globin beta detection primer, right
gtgatcttgaacagagtgtcagtgctga

SEQ ID NO: 27 - DNA, 20 bp, Artificial Sequence
  pSPIKE1 left primer
ctgcattgacggtgctgacc

SEQ ID NO: 28 - DNA, 20 bp, Artificial Sequence
  pSPIKE1 right primer
cggcatcgcattgcccataa

SEQ ID NO: 29 - DNA, 20 bp, Artificial Sequence
  pSPIKE2 left primer
aaggttcccgtgccagttga

SEQ ID NO: 30 - DNA, 20 bp, Artificial Sequence
  pSPIKE2 right primer
gcttctgaaaaccgcctcgg

SEQ ID NO: 31 - DNA, 623 bp, Homo sapiens
cataaaccctggcgcgctcgcggcccggcactcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggccatgcttcttgccccttgggcctccccccagcccctcctccccttcctgcacccgtacccccgtggtctttgaataaagtctgagtgggcggcaaaaaaaaaaaaaaaaaa

SEQ ID NO: 32 - DNA, 622 bp, Homo sapiens
cataaaccctggcgcgctcgcgggccggcactcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagccgttcctcctgcccgctgggcctcccaacgggccctcctcccctccttgcaccggcccttcctggtctttgaataaagtctgagtgggcagcaaaaaaaaaaaaaaaaaa

SEQ ID NO: 33 - DNA, 669 bp, Homo sapiens
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaatcagcactgacactctgttcaagatcac

SEQ ID NO: 34 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnncatgatggg

SEQ ID NO: 35 - DNA, 36 bp, Artificial Sequence
  Barcoded STRT switchign oligonucleotide
  misc_feature (24)..(27): unique molecular identifier
  modified_base (34)..(36): ribose guanine nucleotides
aagcagtggtatcaacgcagagunnnntattgtggg

SEQ ID NO: 36 - DNA, 36 bp, Artificial Sequence
  Barcoded STRT swtiching oligonucleotide
  misc_feature (24)..(27): unique molecular identifier
  modified_base (34)..(36): ribose guanine nucleotides
aagcagtggtatcaacgcagagunnnncgtggcggg

SEQ ID NO: 37 - DNA, 36 bp, Artificial Sequence
  Barcoded STRT switching oligonucleotides
  misc_feature (24)..(27): unique molecular identifier
  modified_base (34)..(36): ribose guanine nucleotides
aagcagtggtatcaacgcagagunnnnaaagttggg

SEQ ID NO: 38 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnntctaccggg

SEQ ID NO: 39 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnntacttcggg

SEQ ID NO: 40 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnncaaagtggg

SEQ ID NO: 41 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnatgcttggg

SEQ ID NO: 42 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngtggtaggg

SEQ ID NO: 43 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngcaggaggg

SEQ ID NO: 44 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnggacatggg

SEQ ID NO: 45 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnaggactggg

SEQ ID NO: 46 - DNA, 36 bp, Artificial Sequence
  Barcoded STRT switching oligonculeotide
  misc_feature (24)..(27): unique molecular identifier
  modified_base (34)..(36): ribose guanine nucleotides
aagcagtggtatcaacgcagagunnnnagtttaggg

SEQ ID NO: 47 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnntgaagcggg

SEQ ID NO: 48 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnntagcatggg

SEQ ID NO: 49 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngttgccggg

SEQ ID NO: 50 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnacgttgggg

SEQ ID NO: 51 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnntaagggggg

SEQ ID NO: 52 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngcctagggg

SEQ ID NO: 53 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnctcgcaggg

SEQ ID NO: 54 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngcgaatggg

SEQ ID NO: 55 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngggtttggg

SEQ ID NO: 56 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngtaatgggg

SEQ ID NO: 57 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnntagagaggg

SEQ ID NO: 58 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnagatggggg

SEQ ID NO: 59 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnatctctggg

SEQ ID NO: 60 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnncgtattggg

SEQ ID NO: 61 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnacttatggg

SEQ ID NO: 62 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngatcttggg

SEQ ID NO: 63 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngctgtgggg

SEQ ID NO: 64 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnacaataggg

SEQ ID NO: 65 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnttcataggg

SEQ ID NO: 66 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnttaactggg

SEQ ID NO: 67 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnatacagggg

SEQ ID NO: 68 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnaataaaggg

SEQ ID NO: 69 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnctgtgtggg

SEQ ID NO: 70 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnncctagaggg

SEQ ID NO: 71 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnccatctggg

SEQ ID NO: 72 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngacactggg

SEQ ID NO: 73 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnntggatgggg

SEQ ID NO: 74 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnctccatggg

SEQ ID NO: 75 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngagatcggg

SEQ ID NO: 76 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnntatgtaggg

SEQ ID NO: 77 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnaacgccggg

SEQ ID NO: 78 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnncttctgggg

SEQ ID NO: 79 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngtcggtggg

SEQ ID NO: 80 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnnatttgcggg

SEQ ID NO: 81 - DNA, 36 bp, Artificial Sequence
  Barcoded STRT switching oligonucleotide
  misc_feature (24)..(27): unique molecular identifier
  modified_base (34)..(36): ribose guanine nucleotides
aagcagtggtatcaacgcagagunnnngaattaggg

SEQ ID NO: 82 - DNA, 26 bp, Artificial Sequence
  Complementary to 3′-end sequence and poly-A tail of human globin alpha mRNA
tttttttgcygcccactcagacttta

SEQ ID NO: 83 - DNA, 27 bp, Artificial Sequence
  Complementary to 3′-end sequence and poly-A tail of human globin alpha mRNA
  modified_base (7)..(7): LNA nucleotide
  modified_base (13)..(13): LNA nucleotide
  modified_base (19)..(19): LNA nucleotide
  modified_base (25)..(25): LNA nucleotide
tttttgcygcccactcagactttattc

SEQ ID NO: 84 - DNA, 45 bp, Artificial Sequence
  Complementary to 3′-end sequence and poly-A tail of human globin alpha mRNA
tttttttttttttttgcygcccactcagactttattcaaagacca

SEQ ID NO: 85 - DNA, 40 bp, Artificial Sequence
  Complementary to 3′-end sequence and poly-A tail of human globin beta mRNA
ttttttttttttgcaatgaaaataaatgttttttattagg

SEQ ID NO: 86 - DNA, 38 bp, Artificial Sequence
  Complementary to 3′-end sequence and poly-A tail of human globin beta mRNA
  modified_base (12)..(12): LNA nucleotide
  modified_base (18)..(18): LNA nucleotide
  modified_base (24)..(24): LNA nucleotide
  modified_base (30)..(30): LNA nucleotide
ttttttttttgcaatgaaaataaatgttttttattagg

SEQ ID NO: 87 - DNA, 55 bp, Artificial Sequence
  Complementary to 3′-end sequence and poly-A tail of human globin beta mRNA
tttttttttttttttgcaatgaaaataaatgttttttattaggcagaatccagat

SEQ ID NO: 88 - DNA, 50 bp, Artificial Sequence
  Complementary to 3′-end sequence and poly-A tail of cow globin alpha mRNA
tttttttttttttttgcagcccactcagactttattcaaagatcaggagg

SEQ ID NO: 89 - DNA, 50 bp, Artificial Sequence
  Complementary to 3′-end sequence and poly-A tail of cow globin beta mRNA
tttttttttttttttgcaatgaaaataaatgtctttattaggcagaggcc

SEQ ID NO: 90 - DNA, 52 bp, Artificial Sequence
  Complementary to 3′-end sequence and poly-A tail of dog globin alpha 1 mRNA
tttttttttttttttgcagcccactcagactttattcaaacatcaggaagtg

SEQ ID NO: 91 - DNA, 40 bp, Artificial Sequence
  Complementary to 3′-end sequence and poly-A tail of dog globin alpha 2 mRNA
tttttttttttttttgggcgcaggaggcggcccagggcag

SEQ ID NO: 92 - DNA, 52 bp, Artificial Sequence
  Complementary to 3′-end sequence and poly-A tail of dog globin beta mRNA
tttttttttttttttgagctgaaggttctttattaggcagaagccataccct

SEQ ID NO: 93 - DNA, 50 bp, Artificial Sequence
  Complementary to 3′-end sequence and poly-A tail of rat globin alpha 1 mRNA
tttttttttttttttgcttcctactcaggctttattcaaagaccaagagg

SEQ ID NO: 94 - DNA, 50 bp, Artificial Sequence
  Complementary to 3′-end sequence and poly-A tail of rat globin alpha 2 mRNA
tttttttttttttttcttcctacttttgctttattcaaagacaggtgcaa

SEQ ID NO: 95 - DNA, 50 bp, Artificial Sequence
  Complementary to 3′-end sequence and poly-A tail of rat globin beta mRNA
tttttttttttttttgcagtgaaagtaaatgccttttattagtaggacag

SEQ ID NO: 96 - DNA, 50 bp, Artificial Sequence
  Complementary to 3′-end sequence and poly-A tail of mouse globin alpha 1 mRNA
tttttttttttttttcttcctactcaggctttattcaaagaccaagaggt

SEQ ID NO: 97 - DNA, 45 bp, Artificial Sequence
  Complementary to 3′-end sequence and poly-A tail of mouse globin alpha 2 mRNA
tttttttttttttttgcaggcttcttcctactcaggctttattca

SEQ ID NO: 98 - DNA, 50 bp, Artificial Sequence
  Complementary to 3′-end sequence and poly-A tail of mouse globin beta mRNA
tttttttttttttttgcagtgaacataaatgctttttatttgtcagaaga

SEQ ID NO: 99 - DNA, 50 bp, Artificial Sequence
  Complementary to 3′-end sequence and poly-A tail of zebrafish globin alpha mRNA
tttttttttttttttagttctgtgccttttattgagaaatttcattcaga

SEQ ID NO: 100 - DNA, 53 bp, Artificial Sequence
  Complementary to 3′-end sequence and poly-A tail of zebrafish globin beta 1 mRNA
tttttttttttttttgcttttaacattattttattgatcttgttgyaaaagag

SEQ ID NO: 101 - DNA, 53 bp, Artificial Sequence; identical to SEQ ID NO: 100
  Complementary to 3′-end sequence and poly-A tail of zebrafish globin beta 2 mRNA

SEQ ID NO: 102 - DNA, 60 bp, Artificial Sequence
  Primer for reverse transcription
  misc_feature (60)..(60): n is a, c, g, or t
ttaagcagtggtatcaacgcagagtcgactttttttttttttttttttttttttttttvn

SEQ ID NO: 103 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnncatgatggg

SEQ ID NO: 104 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnntattgtggg

SEQ ID NO: 105 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnncgtggcggg

SEQ ID NO: 106 - DNA, 34 bp, Artificial Sequence
  Barcoded STRT switching oligonucleotides
  misc_feature (20)..(25): unique molecular identifier
  modified_base (32)..(34): ribose guanine nucleotides
agtggtatcaacgcagagtnnnnnnaaagttggg

SEQ ID NO: 107 - DNA, 34 bp, Artificial Sequence
  Barcoded STRT switching oligonucleotides
  misc_feature (20)..(25): unique molecular identifier
  modified_base (32)..(34): ribose guanine nucleotides
agtggtatcaacgcagagtnnnnnntctaccggg

SEQ ID NO: 108 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnttggacggg

SEQ ID NO: 109 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnncaaagtggg

SEQ ID NO: 110 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnatgcttggg

SEQ ID NO: 111 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnngtggtaggg

SEQ ID NO: 112 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnngcaggaggg

SEQ ID NO: 113 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnggacatggg

SEQ ID NO: 114 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnattccaggg

SEQ ID NO: 115 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnagtttaggg

SEQ ID NO: 116 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnntgaagcggg

SEQ ID NO: 117 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnntagcatggg

SEQ ID NO: 118 - DNA, 34 bp, Artificial Sequence
  Barcoded STRT switching oligonucleotide
  misc_feature (20)..(25): unique molecular identifeir
  modified_base (32)..(34): ribose guanine nucleotides
agtggtatcaacgcagagtnnnnnngttgccggg

SEQ ID NO: 119 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnacgttgggg

SEQ ID NO: 120 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnntaagggggg

SEQ ID NO: 121 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnngcctagggg

SEQ ID NO: 122 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnctcgcaggg

SEQ ID NO: 123 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnngcgaatggg

SEQ ID NO: 124 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnngggtttggg

SEQ ID NO: 125 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnngtaatgggg

SEQ ID NO: 126 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnntagagaggg

SEQ ID NO: 127 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnagatggggg

SEQ ID NO: 128 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnatctctggg

SEQ ID NO: 129 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnncgtattggg

SEQ ID NO: 130 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnacttatggg

SEQ ID NO: 131 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnngatcttggg

SEQ ID NO: 132 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnngctgtgggg

SEQ ID NO: 133 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnacaataggg

SEQ ID NO: 134 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnttcataggg

SEQ ID NO: 135 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnttaactggg

SEQ ID NO: 136 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnatacagggg

SEQ ID NO: 137 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnagtcgtggg

SEQ ID NO: 138 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnctgtgtggg

SEQ ID NO: 139 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnncctagaggg

SEQ ID NO: 140 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnccatctggg

SEQ ID NO: 141 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnngacactggg

SEQ ID NO: 142 - DNA, 34 bp, Artificial Sequence
  Barcoded STRT switching olignucleotide
  misc_feature (20)..(25): unique molecular identifier
  modified_base (32)..(34): ribose guanine nucleotides
agtggtatcaacgcagagtnnnnnntggatgggg

SEQ ID NO: 143 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnctccatggg

SEQ ID NO: 144 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnngagatcggg

SEQ ID NO: 145 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnntatgtaggg

SEQ ID NO: 146 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnaacgccggg

SEQ ID NO: 147 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnncttctgggg

SEQ ID NO: 148 - DNA, 34 bp, Artificial Sequence
  Barcoded STRT switching oligonucelotide
  misc_feature (20)..(25): unique molecular identifier
  modified_base (32)..(34): ribose guanine nucleotides
agtggtatcaacgcagagtnnnnnngtcggtggg

SEQ ID NO: 149 - DNA, 34 bp, Artificial Sequence
  Barcoded STRT switching oligonucleotide
  misc_feature (20)..(25): unique molecular identifier
  modified_base (32)..(34): ribose guanine nucleotides
agtggtatcaacgcagagtnnnnnnatttgcggg

SEQ ID NO: 150 - DNA, 34 bp, Artificial Sequence
  Barcoded STRT switching oligonucleotide
  misc_feature (20)..(25): unique molecular identiifer
  modified_base (32)..(34): ribose guanine nucleotides
agtggtatcaacgcagagtnnnnnncgagtaggg

SEQ ID NO: 151 - DNA, 24 bp, Artificial Sequence
  qPCR cow globin beta detector primer, left
aagtggatgaagttggtggtgagg

SEQ ID NO: 152 - DNA, 24 bp, Artificial Sequence
  qPCR cow globin beta detection primer, right
tccacatgcagcttatcacagtgc

SEQ ID NO: 153 - DNA, 20 bp, Artificial Sequence
  qPCR dog globin alpha detection primer, left
cccgtcaacttcaagctcct

SEQ ID NO: 154 - DNA, 23 bp, Artificial Sequence
  qPCR dog globin alpha detection primer, right
tcttgaacagagtgtcagtgctg

SEQ ID NO: 155 - DNA, 24 bp, Artificial Sequence
  qPCR dog globin beta detection primer, left
gcaatgctaaagtgaaggcccatg

SEQ ID NO: 156 - DNA, 24 bp, Artificial Sequence
  qPCR dog globin beta detection primer, right
caggagcttgaagttctcgggatc

SEQ ID NO: 157 - DNA, 24 bp, Artificial Sequence
  qPCR rat globin alpha detection primer, left
caaactgcgtgtggatcctgtcaa

SEQ ID NO: 158 - DNA, 24 bp, Artificial Sequence
  qPCR rat globin alpha detection primer, right
ggcttaacggtacttggaggtcag

SEQ ID NO: 159 - DNA, 24 bp, Artificial Sequence
  qPCR rat globin beta detection primer, left
gtgcacctgactgatgctgagaag

SEQ ID NO: 160 - DNA, 24 bp, Artificial Sequence
  qPCR rat globin beta detection primer, right
ttgtccaagtgtttcaggccatca

SEQ ID NO: 161 - DNA, 24 bp, Artificial Sequence
  qPCR mouse globin alpha detection primer, left
gatcccgtcaacttcaagctcctg

SEQ ID NO: 162 - DNA, 24 bp, Artificial Sequence
  qPCR mouse globin alpha detection primer, right
aaggcagcttaacggtacttggag

SEQ ID NO: 163 - DNA, 24 bp, Artificial Sequence
  qPCR mouse globin beta detection primer, left
gctcctgggcaatatgatcgtgat

SEQ ID NO: 164 - DNA, 24 bp, Artificial Sequence
  qPCR mouse globin beta detection primer, right
ttgccaacaactgacagatgctct

SEQ ID NO: 165 - DNA, 24 bp, Artificial Sequence
  qPCR zebrafish globin alpha detection primer, left
gcagcgatgagtctctctgatacg

SEQ ID NO: 166 - DNA, 24 bp, Artificial Sequence
  qPCR zebrafish globin alpha detection primer, right
cacccatgatagtctttccgtgct

SEQ ID NO: 167 - DNA, 19 bp, Artificial Sequence
  qPCR zebrafish globin beta detection primer, left
ggaggcctggcagaagttt

SEQ ID NO: 168 - DNA, 23 bp, Artificial Sequence
  qPCR zebrafish globin beta detection primer, right
tcttgaacagagtgtcagtgctg

SEQ ID NO: 169 - DNA, 34 bp, Artificial Sequence
  Complementary to 3′-end sequence and poly-A tail of human globin alpha mRNA
ttttgcygcccactcagactttattcaaagacca

SEQ ID NO: 170 - DNA, 32 bp, Artificial Sequence
  Complementary to 3′-end sequence and poly-A tail of human globin alpha mRNA
ttgcygcccactcagactttattcaaagacca

SEQ ID NO: 171 - DNA, 30 bp, Artificial Sequence
  Complementary to 3′-end sequence of human globin alpha mRNA
gcygcccactcagactttattcaaagacca

SEQ ID NO: 172 - DNA, 28 bp, Artificial Sequence
  Complementary to 3′-end sequence of human globin alpha mRNA
ygcccactcagactttattcaaagacca

SEQ ID NO: 173 - DNA, 26 bp, Artificial Sequence
  Complementary to 3′-end sequence of human globin alpha mRNA
cccactcagactttattcaaagacca